(19) United States
(12) Patent Application Publication
(10) Pub. No.: US 2025/0359771 A1
(43) Pub. Date: Nov. 27, 2025

(54) HEART HEALTH MONITORING USING LASER SPECKLE CONTRAST IMAGING IN A NEAR-EYE DEVICE

(71) Applicant: Meta Platforms Technologies, LLC, Menlo Park, CA (US)

(72) Inventors: Mantas Zurauskas, Redmond, WA (US); Jindou Shi, Redmond, WA (US)

(21) Appl. No.: 19/211,513

(22) Filed: May 19, 2025

Related U.S. Application Data
(60) Provisional application No. 63/651,228, filed on May 23, 2024.

(51) Int. Cl. A61B 5/024; A61B 5/00

(52) U.S. Cl. CPC A61B 5/02427; A61B 5/0075

(57) ABSTRACT

A device for performing a speckle contract imaging operation is provided. The device comprises a frame including a projector and a sensor such that the projector is configured to project a speckle pattern on a facial region of a user, and the sensor is configured to receive reflections of the speckled pattern. Further, the device comprises a controller configured to receive a time series of images of the facial region and perform a speckle contrast imaging operation on the time series of images.

### CROSS REFERENCE TO RELATED APPLICATION

This application claims the benefit of a U.S. Provisional patent application having U.S. Provisional Patent Application No. 63/651,228, filed on May 23, 2024, the disclosure of which is incorporated herein, in its entirety, by this reference.

### TECHNICAL FIELD

This patent application relates generally to monitoring heart health using the capabilities of a near-eye device, and in particular to using eye/face tracking and techniques such as, e.g., Laser Speckle Contrast Imaging (LSCI), for heart health measurement in a near-eye device.

### BACKGROUND

With recent advances in technology, the prevalence of content creation and delivery has increased greatly in recent years. In particular, interactive content such as virtual reality (VR) content, augmented reality (AR) content, mixed reality (MR) content, and/or any other content within and associated with a real and/or virtual environment (e.g., a “metaverse”) has become appealing to consumers. Various forms of wearable content-providing systems may be employed to facilitate content delivery. One such example may be wearable devices, such as a wrist-worn devices, armbands, and/or near-eye devices, i.e., wearable eyewear, which may include wearable headsets (such as, e.g., a head-mounted display (HMO) device), or digital content devices in the form of eyeglasses. In some examples, the near-eye device may be a display device, which may project or direct light to may display virtual objects or combine images of real objects with virtual objects, as in augmented reality (AR), mixed reality (MR), virtual reality (VR) and/or other digital content applications. For example, in a near-eye device having an augmented reality (AR) and/or a mixed reality (MR) system, a user may view both images of virtual objects (e.g., computer-generated images (CGIs)) and the surrounding environment. A near-eye display device may also present interactive content, where a user's (wearer's) gaze may be used as input for modifying, directing, and/or otherwise affecting the interactive content.

The development of health and fitness technology using wearable devices are intertwined with the development of wearable devices capable of providing digital content. In the realm of health and fitness technology, heart rate measurement has become a standard feature, particularly in smartwatches, which typically have a fitness tracker. Such devices, equipped with advanced sensors, provide real-time heart rate data, and may offer valuable insights into an individual's health and fitness levels. Many smartwatches and similar devices may measure heart rate through a process called photoplethysmography, which uses light to measure changes in blood volume in the wrist, which can then be used to calculate heart rate. This technology has changed the way many monitor their health, making it possible to track heart rates over time, identify irregularities, and thus potentially detect serious health conditions. Improvement in heart health monitoring may be beneficial for different heart health monitoring devices.

### BRIEF DESCRIPTION OF THE DRAWINGS

Features of the present disclosure are illustrated by way of example and not limited in the following figures, in which like numerals indicate like elements. One skilled in the art will readily recognize from the following that alternative examples of the structures and methods illustrated in the figures can be employed without departing from the principles described herein;

FIG. 1 illustrates a block diagram of a near-eye device system which may form part of a display system environment, according to an example;

FIGS. 2A and 2B illustrate a front prospective view and a back prospective view, respectively, of a near-eye display device in the form of a head-mounted display (HMO) device to which examples of the present disclosure may be applied;

FIGS. 3A and 3B illustrate a perspective view and a top view, respectively, of a near-eye display device in the form of a pair of glasses to which examples of the present disclosure may be applied;

FIGS. 4A and 4B illustrate eye vasculature visualization using speckle contrast imaging, which may be employed in accordance with examples of the present disclosure;

FIGS. 5A and 5B illustrate the measurement of heart rate based on blood flow pulses in eye capillaries, which may be employed in accordance with examples of the present disclosure;

FIG. 6 is a block diagram of an eye-face tracking system in a near-eye device, with a global shutter eye-face tracking camera for health monitoring, according to an example of the present disclosure;

FIG. 7 is a block diagram of an eye-face tracking system in a near-eye device, with a rolling shutter eye-face tracking camera for health monitoring, according to an example of the present disclosure;

FIGS. 8A and 8B are block diagrams of an eye-face tracking system in a near-eye device, in which an eye-face tracking camera is pointed into a waveguide/display of the near-eye device and is capable of health monitoring, according to examples of the present disclosure;

FIGS. 9A and 9B are block diagrams of an eye-face tracking system in a near-eye device, in which the eye-face tracking sensors/projectors consist of an array of Self-Mixing Interferometric Vertical Cavity Side Emitting Lasers (SMINCSELs) which are capable of health monitoring, according to examples of the present disclosure;

FIG. 10 is a block diagram of an eye-face tracking system in a near-eye device, in which the eye-face tracking camera is a high-resolution camera capable of health monitoring, according to examples of the present disclosure;

FIG. 11 is a flowchart illustrating a method for an eye/face tracking system in a near-eye device to perform health monitoring using speckle contrast imaging, according to examples of the present disclosure;

FIG. 12 is a flow diagram for a method of health monitoring using speckle contrast imaging by an eye/face tracking system in a near-eye device, according to examples of the present disclosure; and

FIGS. 13A-13C are flowcharts illustrating methods for heart rate measurement employing an eye/face tracking system in a near-eye device which performs speckle contrast imaging, according to examples of the present disclosure.

### DETAILED DESCRIPTION

For simplicity and illustrative purposes, the present application is described by referring mainly to examples thereof. In the following description, numerous specific details are set forth in order to provide a thorough understanding of the present application. It will be readily apparent, however, that the present application may be practiced without limitation to these specific details. In other instances, some methods and structures readily understood by one of ordinary skill in the art have not been described in detail so as not to unnecessarily obscure the present application. As used herein, the terms “a” and “an” are intended to denote at least one of a particular element, the term “includes” means includes but not limited to, the term “including” means including but not limited to, and the term “based on” means based at least in part on.

As used herein, a “wearable device” may refer to any portable electronic device that may be worn on any body part of a user and used to present audio and/or video content, control other devices, monitor bodily functions, and/or perform similar actions.

As used herein, a “near-eye device” may refer to a device that may be in close proximity to a user's eye and may have optical and computing capabilities, whereas a “near-eye display device” may refer to a device that may be in close proximity to a user's eye and may be capable of some sort of display to one or both of the user's eyes. In some examples, a near-eye device may be “smartglasses” in the form of a pair of normal eyeglasses, and/or a wearable headset, such as a head-mounted display (HMO) device, and may have auxiliary operatively connected equipment (which may be wired and/or wirelessly connected), such as a handset, wristband, input/output (I/O) controller, computer “puck,” etc. In some examples, a near-eye display device may display visual content; in some examples, the visual content may include virtual reality (VR), augmented reality (AR), and/or mixed reality (MR) content, and/or may include an interactive virtual reality (VR), augmented reality (AR), and/or mixed reality (MR) environment, or any artificial reality environment which includes real and/or virtual elements, such as a “metaverse.”

As used herein, a “near-eye VR/AR/MR display device” may refer to a near-eye display device which may be used to display and/or for interact with any virtual reality (VR), augmented reality (AR), and/or mixed reality (MR) content, including, but not limited to, any interactive virtual reality (VR), augmented reality (AR), and/or mixed reality (MR) environment (or metaverse). As used herein, a “user” may refer to a user or wearer of a “wearable device,” “near-eye device,” “near-eye display device,” and/or “near-eye VR/AR/MR display device,” depending on the context, which would be clear to one of ordinary skill in the art.

As mentioned above, improvements to health monitoring may be beneficial in all types of wearable devices, as heart rate measurements are one of the key metrics that may be collected by wearable devices. For near-eye VR/AR/MR display devices, in addition to health and fitness monitoring, the incorporation of heart rate measurement capabilities may provide valuable insights into a user's emotional and physiological state, which can enhance the VR/AR/MR experience by enabling more personalized and immersive interactions. However, there may be a challenge in developing heart health monitoring techniques which are compatible with the unique form factor of near-eye VR/AR/MR display devices. Moreover, there may be a challenge of the additional cost, use of resources, use of additional power/energy, etc., for adding heart monitoring hardware to near-eye VR/AR/MR display devices.

According to examples of the present disclosure, the pre-existing eye/face tracking system in a near-eye device may be employed for health monitoring. In some examples, the eye/face tracking system may include an eye/face tracking projector to project a speckle pattern on a user's eye and/or surrounding facial tissue; an eye/face tracking sensor to receive reflections of the speckled pattern from the user's eye and/or surrounding facial tissue; and an eye/face tracking controller to receive a time series of frames/images of the user's eye and/or surrounding facial tissue generated from eye/face tracking sensor data, to perform speckle contrast imaging (e.g., a speckle contrast image operation) on the received time series of frames/images, and to perform health monitoring using results of the speckle contrast imaging.

In some examples, the eye/face tracking sensor may include a global shutter camera or a rolling shutter camera. In some examples, the eye/face tracking sensor may point roughly in the direction of the user's eye and/or surrounding facial tissue; in other examples, the eye/face tracking sensor may be disposed to receive reflections through a waveguide/display of the near-eye device by internal reflection (IR). In some examples, the eye/face tracking sensor may be a high-resolution camera capable of detecting microtremors in the user's eye and/or surrounding facial tissue, which may be employed to calculate a heart rate of the user. In such examples, ambient lighting may be used rather than projected sparkle patterns.

In some examples, the eye/face tracking projector may be any suitable coherent light source. In some examples, the eye/face tracking projector may be disposed on the front frame of the near-eye device and may face roughly in the direction of the user's eye and/or surrounding facial tissue; in some examples, the eye/face tracking projector may be disposed to project speckle patterns through a waveguide/display of the near-eye device by internal reflection (IR); in other examples, the eye/face tracking projector may be disposed on the temple of the near-eye device to project speckle patterns onto a hot mirror embedded in a waveguide/eye lens of the near-eye device to thereby reflect the speckle patterns into the user's eye and/or surrounding facial tissue.

In some examples, the eye/face tracking projector and the eye/face tracking sensor may be combined in the same module or integrated circuit. In some examples, the eye/face tracking projector/sensor may include an array of Self-Mixing Interferometric Vertical Cavity Side Emitting Lasers (SMINCSELs) which may be disposed to project/sense through a waveguide of the near-eye device or may be embedded in a waveguide/eye lens of the near-eye device to face roughly in the direction of the user's eye and/or surrounding facial tissue.

In some examples, the speckle contrast imaging operation may be Laser Speckle contrast Imaging (LSCI) and/or Laser Contrast Imaging (LCI). In some examples, the health monitoring may be of the cardiac function (e.g., cardiovascular parameter) of the user, such as, for example, the heart rate, pulse, and so forth, of the user.

Although the discussions and descriptions herein may sometimes focus on near-eye VR/AR/MR display devices, the present disclosure is not limited thereto and may also be employed in near-eye devices without VR/AR/MR display capabilities, as well as near-eye devices without any display capabilities (which employ an eye-face tracking system for purposes besides display).

While some advantages and benefits of the present disclosure are discussed herein, there are additional benefits and advantages which would be apparent to one of ordinary skill in the art.

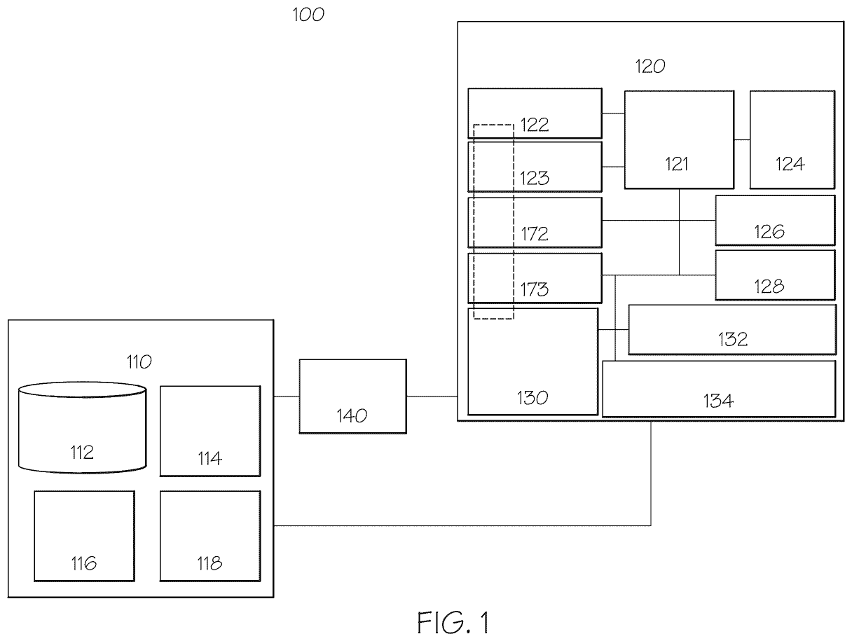

FIG. 1 illustrates a block diagram of a near-eye device system which may be part of an artificial reality display system environment, according to an example. As used herein, a “near-eye device system” may refer to any system including a near-eye device, which may or may not also include separate yet operatively connected equipment (which may be wired and/or wirelessly connected), such as a handset, wristband, input/output (I/O) controller, computer “puck,” sensor, etc. As mentioned above, a “near-eye device” may refer to a device that may be in close proximity to a user's eye and may have optical and computing capabilities, and a “near-eye display device” may refer to a near-eye device capable of some sort of display to one or both of the user's eyes. As also mentioned above, a near-eye device may be “smartglasses” in the form of a pair of eyeglasses, and/or a wearable headset, such as a head-mounted display (HMO) device, and, if it is a near-eye display device, it may be capable of displaying visual content, including, e.g., virtual reality (VR), augmented reality (AR), and/or mixed reality (MR) content. As used herein, a “near-eye VR/AR/MR display device” may refer to a near-eye display device which may be used to display and/or for interact with any virtual reality (VR), augmented reality (AR), and/or mixed reality (MR) content, including, but not limited to, a near-eye display device which may provide any interactive virtual reality (VR), augmented reality (AR), and/or mixed reality (MR) environment (or metaverse) to its user. As used herein, a “VR/AR/MR” may refer to any one or more of virtual reality (VR) content, augmented reality (AR) content, and/or mixed reality (MR) content, and accordingly may include any interactive virtual reality (VR), augmented reality (AR), and/or mixed reality (MR) environment (or metaverse), depending on the context, which would be understood by one of ordinary skill in the art. As used herein, a “user” may refer to a user or wearer of a “wearable device,” “near-eye device,” “near-eye display device,” and/or “near-eye VR/AR/MR display device,” depending on the context, which would be understood by one of ordinary skill in the art.

While this section describes near-eye display device systems, examples of the present disclosure are not limited thereto. For instance, examples of the present disclosure may apply to near-eye devices without specific image displaying capabilities, such as, for example, the Ray-Ban™|Meta™ line of smartglasses. Moreover, examples of the present disclosure are expressly intended to apply to other wearable devices (as defined above) besides the near-eye devices described herein, including other wearable computing platforms, which may have, e.g., Internet of Things (IoT), audio/visual, health monitoring, WiFi and radio reception, and/or other capabilities, such as smartwatches, compute “pucks,” as would be understood by one of ordinary skill in the art.

As shown in FIG. 1, an artificial reality system 100 may include a near-eye display device 120 and an optional input/output interface 140, each of which may be coupled to an optional console 110, where the artificial reality system 100 may or may not be virtual reality (VR), augmented reality (AR), and/or mixed reality (MR) system which may, or may not, display images or other content to the user. The artificial reality system 100 may also include an optional external imaging device (not shown), as discussed in relation to the one or more locators 126 below. As would be understood by one of ordinary skill in the art, FIG. 1 is a schematic diagram, and is not indicative of size, location, orientation, and/or relative sizes/locations/orientations of any of the systems, components, and/or connections shown therein. For example, a figurative “bus” connects some, but not all, of the components shown inside the near-eye display device 120 in FIG. 1; however, all of the components therein may be connected by the same bus and/or busses, or may have direct and/or indirect connections with, e.g., the one or more processors 121. Such electrical, control, and/or power connections may be implemented in a large variety of ways, as would be understood by one of ordinary skill in the art.

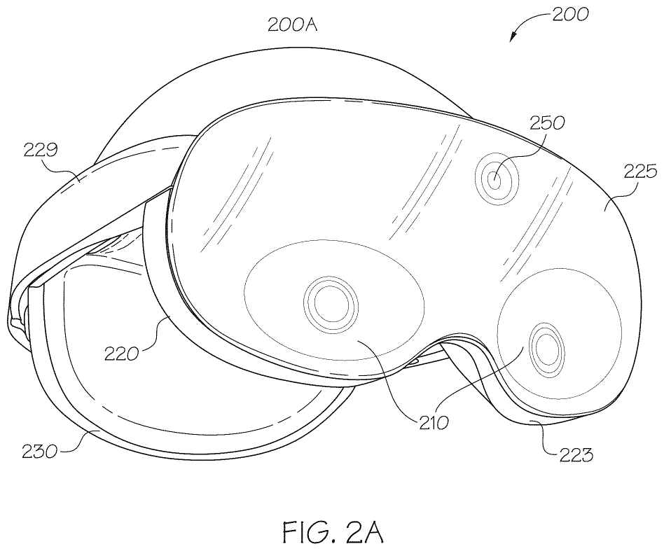

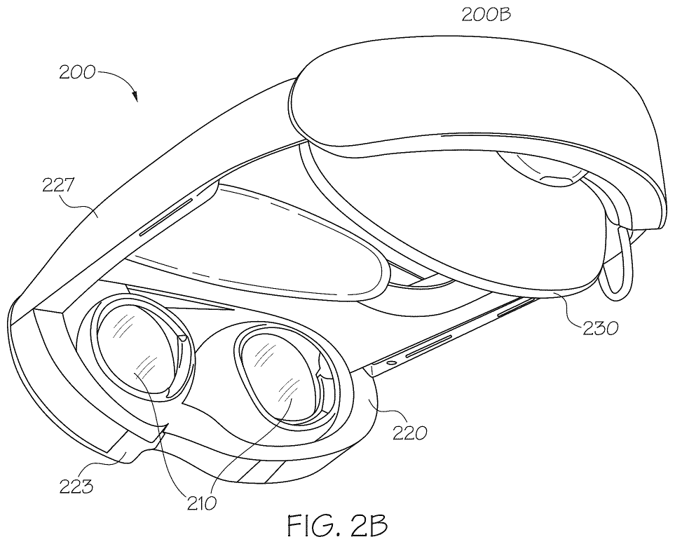

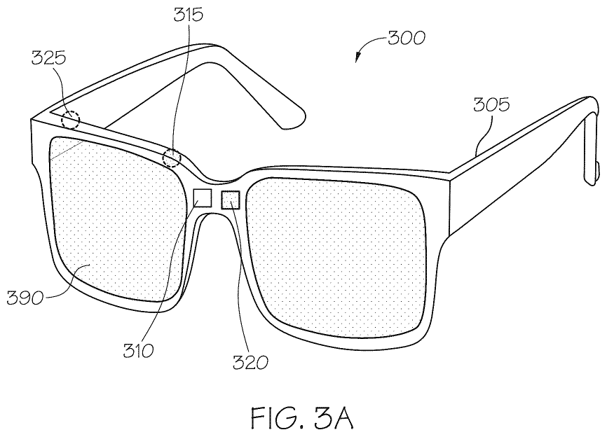

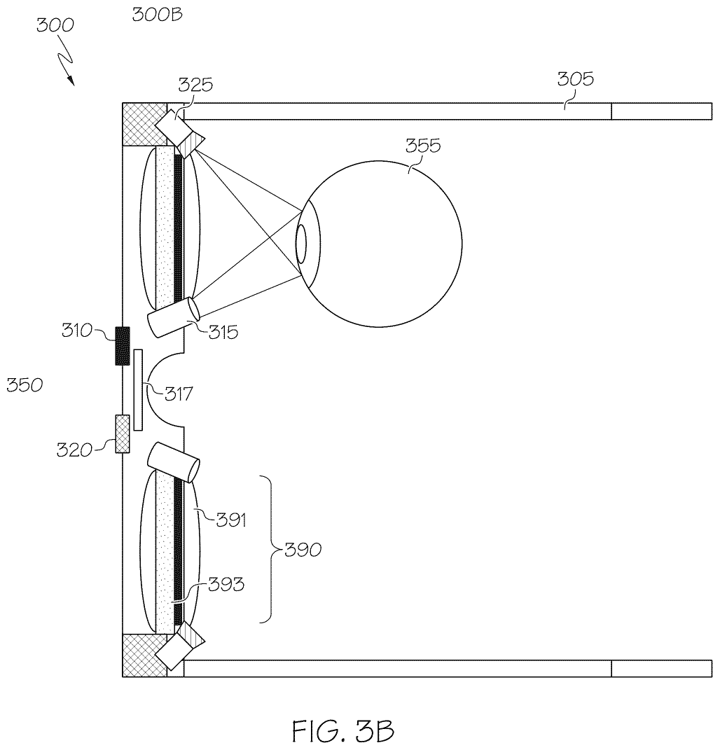

The optional console 110 may be optional in some instances in which functions of the optional console 110 may be integrated into the near-eye display device 120. In some examples, the near-eye display device 120 may be implemented in any suitable form-factor, including a head-mounted display (HMO), a pair of glasses, or other similar wearable eyewear or device. In some examples, the near-eye display device 120 may include one or more rigid bodies, which may be rigidly or non-rigidly coupled to each other. In some examples, a rigid coupling between rigid bodies may cause the coupled rigid bodies to act as a single rigid entity, while in other examples, a non-rigid coupling between rigid bodies may allow the rigid bodies to move relative to each other. Some non-limiting specific examples of implementations of the near-eye display device 120 are described further below with respect to FIGS. 2A-2B and 3A-3B.

In some examples, the near-eye display device 120 may present content to a user, including, for example, audio/visual content, such as, e.g., music or personal communications (e.g., a telephone call) through speakers/microphones, virtual reality (VR), augmented reality (AR), and/or mixed reality (MR) content through displays, etc. In augmented reality (AR) examples, the near-eye display device 120 may combine images (and/or a see-through view) of a physical, real-world environment external to the near-eye display device 120 and artificial reality/digital content (e.g., computer-generated images, video, sound, etc.) to present an augmented reality (AR) environment for the user.

As shown in FIG. 1, the near-eye display device 120 may include any one or more of the one or more processors 121, display electronics 122, one or more outward-facing sensor(s) 123, display optics 124, the one or more locators 126, one or more position sensors 128, an eye/face tracking unit 130, an inertial measurement unit (IMU) 132, a wireless communication subsystem 134, one or more outward projectors 172, and/or the one or more inward projectors 173. In some examples, the near-eye display device 120 may include additional components; in other examples, the near-eye display device 120 may omit any one or more of the one or more locators 126, the one or more position sensors 128, the eye/face tracking unit 130, the inertial measurement unit (IMU) 132, the wireless communication subsystem 134, the one or more outward projectors 172, and/or the one or more inward projectors 173. As would be understood by one of ordinary skill in the art, various operational, electronic, communication (for, e.g., control signals), electrical and other such connections may or may not also be included between and among the components of the near-eye display device 120.

In some examples, the display electronics 122 may display or facilitate the display of images to the user according to data received from control electronics disposed in, for example, the near-eye display device 120, the optional console 110, the input/output interface 140, and/or a system connected by wireless or wired connection with the near-eye display device 120. In some examples, such electronics may include an artificial environment engine, such as, for example, a virtual reality (VR), augmented reality (AR), and/or mixed reality (MR) content (VR/AR/MR) engine 116 in the optional console 110 described below; a VR/AR/MR engine implemented, in part or in whole, in electronics in the near-eye display device 120; and/or a VR/AR/MR engine implemented, in whole or in part, in an external system connected by the wireless communication subsystem 134, etc. In some examples, the display electronics 122 may include one or more display panels, and may include and/or be operationally connected to the display optics 124. In some examples, the display electronics may include one or more of a liquid crystal display (LCD) and/or a light-emitting diode (LED) and may include any number of pixels to emit light of a predominant color such as red, green, blue, white, or yellow. In some examples, the display electronics 122 may display a three-dimensional (3D) image, e.g., using stereoscopic effects produced by two-dimensional panels, to create a subjective perception of image depth.

In some examples, the display electronics 122 may include and/or be operationally connected to the one or more outward projectors 172 and/or the one or more inward projectors 173; in some examples, the eye/face tracking unit 130 may also include and/or be operationally connected to the one or more inward projectors 173. As indicated by the striped lined box in FIG. 1, there may be operational and/or other connections between and among the display electronics 122, the eye/face tracking unit 130, the one or more outward projectors 172, and/or the one or more inward projectors 173. As indicated above, such connections may also be included between and among these and other components of the near-eye display device 120; the possible connections indicated by the striped lined box in FIG. 1 are shown herein as they are germane to examples of the present disclosure.

In some examples, the one or more outward-facing sensor(s) 123 may include, e.g., a camera, an image sensor, such as a complementary metal-oxide semiconductor (CMOS) image sensor, a defocused image sensor, a light field sensor, a single photon avalanche diode (SPAD), and/or, in certain implementations, a non-imaging sensor, such as a self-mixing interferometer (SMI) sensor. In some examples, the one or more outward-facing sensor(s) 123 may be a combined VCSEL/SMI integrated circuit which may be employed as both a light source and a sensor. In some examples, the one or more outward-facing sensor(s) 123 may be employed for purposes of creating a user-responsive VR/AR/MR display environment by sensing the external environment in relation to the user, such as in, an example outward-facing camera 250 in the head-mounted display (HMO) device 200 in FIGS. 2A-2B and/or the outward-facing camera(s) 320 in FIGS. 3A-3B as discussed and described more fully below.

In some examples, the one or more inward projectors 173 may, under the control of the display electronics 122, form an image in angular domain for direct observation by a viewer's eye through a pupil. In some examples, the same or different one or more inward projectors 173 may, under the control of the eye/face tracking unit 130, project a fringe or other pattern on the eye and/or other portions of the user's face (such as the one or more inward projectors 173 of FIGS. 3A and 3B discussed below). As used herein, “eye/face tracking” may refer to determining an eye's position or relative position, including orientation, location, and/or gaze of a user's eye, as well as determining facial characteristics and parameters, such as from the flesh covering the orbital socket, the eyelids, eye brows, and/or any other regions around the eye or optionally elsewhere on the face. In examples where at least some of the one or more inward projectors 173 may be used to project a fringe pattern on the eye and/or face, reflections from the projected pattern on the eye may be captured by a camera and analyzed (e.g., by the eye/face tracking unit 130 and/or the eye/face tracking module 118 in the optional console 110) to determine a position of the eye (the pupil), a gaze, etc., and/or characteristics of one or more portions of the face (including the region immediately adjacent to the eye). In other examples, the eye/face tracking unit 130 may capture reflected radio waves emitted by a miniature radar unit. These data associated with the eye and/or face may be used to determine or predict eye position, orientation, movement, location, gaze, etc., and/or characteristics of one or more portions of the face (including the region immediately adjacent to the eye).

In some examples, the one or more outward projectors 172 may, under the control of the display electronics 122, project a fringe or other pattern on the external environment (such as the one or more outward pattern projectors 310 of FIGS. 3A and 3B). In examples where at least some of the one or more outward projectors 172 may be used to project a fringe pattern on the external environment, reflections from the projected pattern on the external environment may be captured by a camera and analyzed to determine a position of objects in the external environment, distances between the user and objects and/or surfaces of the external environment, etc.

In some examples, a location of any of the one or more inward projectors 173 and/or the one or more outward projectors 172 may be adjusted to enable any number of design modifications. For example, in some instances, the one or more inward projectors 173 may be disposed in the near-eye display device 120 in front of the user's eye (e.g., “front-mounted” placement). In a front-mounted placement, in some examples, the one or more inward projectors 173 under control of the display electronics 122 may be located away from a user's eyes (e.g., “world-side”). In some examples, the near-eye display device 120 may utilize a front-mounted placement to propagate light and project an image on the user's eye(s).

In some examples, the one or more outward projectors 172 and/or the one or more inward projectors 173 may employ a controllable light source (e.g., a laser) and a micro-electromechanical system (MEMS) beam scanner to create a light field from, for example, a collimated light beam. In some examples, the light source of the one or more outward projectors 172 and/or the one or more inward projectors 173 may include one or more of a Vertical Cavity Surface Emitting Laser (VCSEL), liquid crystal display (LCD), a light emitting diode (LED) or micro-light emitting diode (mLED), an organic light emitting diode (OLEO), an inorganic light emitting diode (ILED), an active-matrix organic light emitting diode (AMOLED), a transparent organic light emitting diode (TLED), any other suitable light source, and/or any combination thereof. In some examples, the one or more projectors (the one or more outward projectors 172 or the one or more inward projectors 173) may be a part of a single electronic display or multiple electronic displays (e.g., one for each eye of the user).

In some examples, the display optics 124 may project, direct, and/or otherwise display image content optically and/or magnify image light received from the one or more inward projectors 173 (and/or otherwise created by the display electronics 122), correct optical errors associated with image light created and/or received from the external environment, and/or present the (corrected) image light to a user of the near-eye display device 120. In some examples, the display optics 124 may include an optical element or any number of combinations of various optical elements as well as mechanical couplings to, for example, maintain relative spacing and orientation of the optical elements in the combination.

In some examples, the display optics 124 may include one or more of a beamforming element, a beam-shaping element, an aperture, a Fresnel lens, a refractive element (such as, e.g., a lens), a reflective element (such as, e.g. a mirror), a diffractive element, a polarization element, a waveguide, a filter, or any other optical element suitable for affecting and/or otherwise manipulating light emitted from the one or more inward projectors 173 (and/or otherwise created by the display electronics 122). In some examples, the display optics 124 may include an optical coating, such as an anti-reflective coating, a reflective coating, a filtering coating, and/or a combination of different optical coatings. In some examples, the display optics 124 may include a Pancharatnam-Berry phase (PBP) or other phase-modification elements, a surface grating, a high-contrast grating, diffractive gratings (such as, e.g. Polarization Volumetric Hologram-based (PVH) gratings, Surface Relief Gratings (SRGs), Volume Bragg Gratings (VBGs), a diffractive optical element (DOE), etc.), nano-optics (including, e.g., metalenses and metasurfaces), micro-structures (including those fabricated using 3D printing), a liquid lens, a mask (such as, e.g., a phase mask), surface coatings, lithographically-created layered waveguides, and/or any other suitable technology, layer, coating, and/or material feasible and/or possible either presently or in the future, as would be understood by one of ordinary skill in the art.

In some examples, the display optics 124 may be used to combine the view of an environment external to the near-eye display device 120 and artificial reality content (e.g., computer-generated images) generated by, e.g., the VR/AR/MR engine 116 in the optional console 110, and projected by, e.g., the one or more inward projectors 173 (and/or otherwise created by the display electronics 122). In such examples, the display optics 124 may augment images of a physical, real-world environment external to the near-eye display device 120 with generated and/or overlaid digital content (e.g., images, video, sound, etc.) projected by the one or more inward projectors 173 (and/or otherwise created by the display electronics 122) to present augmented reality (AR) content to a user.

In some examples, the display optics 124 may also be designed to correct one or more types of optical errors, such as two-dimensional optical errors, three-dimensional optical errors, or any combination thereof. Examples of two-dimensional errors may include barrel distortion, pincushion distortion, longitudinal chromatic aberration, and/or transverse chromatic aberration. Examples of three-dimensional errors may include spherical aberration, chromatic aberration field curvature, and astigmatism.

In some examples, the one or more locators 126 may be objects located in specific positions relative to one another and relative to a reference point on the near-eye display device 120. In some examples, the optional console 110 may identify the one or more locators 126 in images captured by an optional external imaging device to determine the artificial reality headset's position, orientation, or both. The one or more locators 126 may each be a light-emitting diode (LED), a corner cube reflector, a reflective marker, a type of light source that contrasts with an environment in which the near-eye display device 120 operates, or any combination thereof.

In some examples, the optional external imaging device (not shown) may include one or more cameras, one or more video cameras, any other device capable of capturing images including the one or more locators 126, or any combination thereof. The optional external imaging device may detect light emitted or reflected from the one or more locators 126 in a field of view of the optional external imaging device.

In some examples, the one or more position sensors 128 may sense motion of the near-eye display device 120 and, in response, generate one or more measurement signals and/or data. Examples of the one or more position sensors 128 may include any number of accelerometers, gyroscopes, magnetometers, and/or other motion-detecting or error-correcting sensors, or any combination thereof.

In some examples, the inertial measurement unit (IMU) 132 may be an electronic device that generates fast calibration data based on measurement signals received from the one or more position sensors 128. The one or more position sensors 128 may be located external to the inertial measurement unit (IMU) 132, internal to the inertial measurement unit (IMU) 132, or any combination thereof. Based on the one or more measurement signals from the one or more position sensors 128, the inertial measurement unit (IMU) 132 may generate fast calibration data indicating an estimated position of the near-eye display device 120. Estimated positions may be of a reference point on the near-eye display device 120, and estimated positions may be, for example, relative to an initial position of the near-eye display device 120, relative to other objects in an external environment, relative to virtual objects in an artificial environment or augmented/mixed reality, etc., as would be understood by one of ordinary skill in the art. For example, the inertial measurement unit (IMU) 132 may integrate measurement signals received from accelerometers over time to estimate a velocity vector and integrate the velocity vector over time to determine an estimated position of the near-eye display device 120. Alternatively, the inertial measurement unit (IMU) 132 may provide the sampled measurement signals to the optional console 110, which may determine the fast calibration data.

In some examples, the wireless communication subsystem 134 may include an ultra wide band (UWB) transceiver. Ultra-wide band (UWB) wireless communication technology is used for short-range, fast, and secure data transmission environments. Ultra-wide band (UWB) wireless communication technology provides high transmission speed, low power consumption, and large bandwidth, in addition to the ability to co-exist with other wireless transmission technologies. The ultra-wide band (UWB) transceiver may be used to detect another user (head-mounted display (HMO) device) within range of communication and within an angle-of-arrival (AoA), then establish line-of-sight (LoS) communication between the two users. The communication may be in audio mode only or in audio/video mode. In other examples, the ultra-wide band (UWB) transceiver may be used to detect the other user, but a different communication technology (transceiver) such as WiFi or Bluetooth Low Energy (BLE) may be used to facilitate the line-of-sight (LOS) communication. In some examples, the wireless communication subsystem 134 may include one or more global navigation satellite system (GNSS) receivers, such as, e.g., a global positioning service (GPS) receiver, one or more transceivers compliant with the Institute of Electrical & Electronic Engineers (IEEE) 803.11 family of present and/or future standards (such as, e.g., “WiFi”), one or more Bluetooth transceivers, one or more cellular receivers and/or transmitters (compliant with any of the 3^{rd} Generation Partnership Project (3GPP), Open Radio Access Network (O-RAN), evolved Common Public Radio Interface (eCPRI), etc., standards), and/or any other receiver and/or transmitter compliant with any suitable communication protocol (also including any unnamed protocols, such as WiMax, NearLink, Zigbee, etc., that would be known to one of ordinary skill in the art). In some instances, any of these communication transceivers may also be implemented in other suitable components of the near-eye display device 120, Input/output interface 140, and/or the optional console 110.

In some cases, multiple wireless communication transceivers may be available for, inter alia, the wireless communication subsystem 134 and/or other components of the artificial reality system 100, and the one with lowest power consumption, highest communication quality (e.g., based on interfering signals), or user choice may be used. For example, the communication technology may be selected based on a lowest power consumption for a given range. In some examples, the one or more processors 121 may be the control electronics (which may include, e.g., an operating system) for the near-eye display device 120. The one or more processors 121 may be employed for controlling one or more of the display electronics 122, the display optics 124, the one or more locators 126, the one or more position sensors 128, the eye/face tracking unit 130, the inertial measurement unit (IMU) 132, the wireless communication subsystem 134, the one or more outward projectors 172, and/or the one or more inward projectors 173, according to the present disclosure. The one or more processors 121 may be implemented, in whole or in part, as a separate physical component in the near-eye display device 120, as distributed among and/or integrated into one or more components of the near-eye display device 120 (such as, e.g., the display electronics 122), and/or externally to near-eye display device 120, such as being implemented/integrated in, for example, the input/output interface 140 and/or the optional console 110 (e.g., the eye/face tracking module 118, the headset tracking module 114, the VR/AR/MR engine 116, the application store 112, etc.), and/or in another external system connected by, for example, the wireless communication subsystem 134. In some examples, the one or more processors 121 of the near-eye display device 120 may receive input, store, and process data, and/or control the components of the near-eye display device 120 in accordance with received input and/or stored/processed data in order to maintain optimal operating conditions of one or more components in the near-eye display device 120.

In some examples, the one or more processors 121, any control electronics, and/or any of the other components of the near-eye display device 120 may be implemented in and/or by any number of processors executing instructions stored on any number of non-transitory computer-readable storage media (not shown) disposed on/in and/or communicatively linked to the near-eye display device 120. The one or more processors 121 may include multiple processing units executing instructions in parallel. The non-transitory computer-readable storage medium/media may be any memory, such as a hard disk drive, a removable memory, or a solid-state drive (e.g., flash memory or dynamic random access memory (DRAM)). In some examples, the one or more processors 121 in the near-eye display device 120 may perform one or more functions; in some examples, one or more non-transitory computer-readable storage media in the near-eye display device 120 may store instructions that, when executed by the one or more processors 121, cause the one or more processors 121 to perform any of the functions described herein and/or to control any of the components described herein. In some examples, functions such as those described below in reference to the optional console 110 (e.g., eye/face tracking, headset tracking, and the generation of virtual reality images) may be performed by the one or more processors 121 integrated with and/or wired/wirelessly connected to the near-eye display device 120.

In some examples, the input/output interface 140 may be a device that allows a user to send action requests to the optional console 110 and/or the near-eye display device 120. As used herein, an “action request” may be a request to perform a particular action. For example, an action request may be to start or to end an application or to perform a particular action within the application. The input/output interface 140 may include one or more input devices. Example input devices may include a keyboard, a mouse, a game controller, a glove, a button, a touch screen, or any other suitable device for receiving action requests and communicating the received action requests to the optional console 110. In some examples, an action request received by the input/output interface 140 may be communicated to the optional console 110 and/or the near-eye display device 120, either or both of which may perform an action corresponding to the requested action.

In some examples, the optional console 110 may provide content to the near-eye display device 120 for presentation to the user in accordance with information received from one or more of the near-eye display device 120, the input/output interface 140, and/or the external imaging device. For example, as shown in the example of FIG. 1, the optional console 110 may include an application store 112, a headset tracking module 114, a VR/AR/MR engine 116, and an eye/face tracking module 118. In some examples, the optional console 110 may include different or additional modules than those described herein, and the functions described further below may be distributed among the components of the optional console 110 in a different manner than is described here (or may be distributed, in part or whole, in one or more components in the near-eye display device 120). It should be appreciated that the optional console 110 may or may not be needed, or the optional console 110 may be integrated, in whole or in part, with the input/output interface 140 and/or the near-eye display device 120, or the optional console 110 may be separate from the input/output interface 140 and/or the near-eye display device 120. In some examples, the optional console 110 may include a processor and a non-transitory computer-readable storage medium storing instructions executable by the processor (including, for example, the application store 112).

In some examples, the application store 112 may store one or more applications for execution by one or more processors in any one or more of the optional console 110, the near-eye display device 120, the input/output interface 140, and/or the optional external imaging device. An application may include a group of instructions that, when executed by a processor, generates content for presentation to the user. Examples of the applications may include gaming applications, conferencing applications, video playback application, or other suitable applications.

In some examples, the VR/AR/MR engine 116 may execute applications within the artificial reality system 100 and receive position/acceleration/velocity information of the near-eye display device 120, predicted future positions of the near-eye display device 120, or any combination thereof from the headset tracking module 114. In some examples, the VR/AR/MR engine 116 may also receive estimated eye position and orientation information from the eye/face tracking module 118. Based on the received information, the VR/AR/MR engine 116 may determine content including, e.g., virtual reality images, to provide to the near-eye display device 120 for presentation to the user.

In some examples, the eye/face tracking module 118, which may be implemented as a processor, may receive eye/face tracking data from the eye/face tracking unit 130 and determine, for example, the position of the user's eye based on the eye/face tracking data. In some examples, the position of the eye may include an eye's orientation, location, or both relative to the near-eye display device 120 or any element thereof. Accordingly, in these examples, because the eye's axes of rotation change as a function of the eye's location in its socket, determining the eye's location in its socket may allow the eye/face tracking module 118 to determine the eye's orientation with increased accuracy.

Generally speaking, any one or more components shown in FIG. 1 may be further broken down into sub-components and/or combined together to form larger modules, as would be understood by one of ordinary skill in the art. For example, in some examples, the near-eye display device 120 may include additional, fewer, and/or different components than shown and/or described in reference to FIG. 1. Moreover, groupings of components may work together as subsystems within the near-eye display device 120, and/or share/provide/transmit data and/or control information, etc., as would be understood by one of ordinary skill in the art. For example, as indicated by the dotted line box connecting/overlapping the display electronics 122, the one or more outward-facing sensor(s) 123, the one or more outward projectors 172, the one or more inward projectors 173, and the eye/face tracking unit 130 in FIG. 1, these listed components may work together and/or may be somewhat integrated in terms of form and/or function in actual implementations of the near-eye display device 120 in FIG. 1.

Generally speaking, any one or more of the components and/or functionalities described in reference to any of the drawings/figures herein may be implemented by hardware, software, and/or any combination thereof, according to examples of the present disclosure. In some examples, the components and/or functionalities may be implemented by any type of application, program, library, script, task, service, process, or any type or form of executable instructions executed on hardware such as circuitry that may include digital and/or analog elements (e.g., one or more transistors, logic gates, registers, memory devices, resistive elements, conductive elements, capacitive elements, and/or the like, as would be understood by one of ordinary skill in the art). In some examples, the hardware and data processing components used to implement the various processes, operations, logic, and circuitry described in connection with the examples described herein may be implemented with a general purpose single- and/or multi-chip processor, a single- and/or multi-core processor, a digital signal processor (DSP), an application specific integrated circuit (ASIC), a field programmable gate array (FPGA), or other programmable logic device, discrete gate or transistor logic, discrete hardware components, and/or any combination thereof suitable to perform the functions described herein. A general purpose processor may be any conventional processor, microprocessor, controller, microcontroller, and/or state machine. In some examples, the memory/storage may include one or more components (e.g., random access memory (RAM), read-only memory (ROM), flash or solid state memory, hard disk storage, etc.) for storing data and/or computer-executable instructions for completing and/or facilitating the processing and storage functions described herein. In some examples, the memory/storage may be volatile and/or non-volatile memory, and may include database components, object code components, script components, or any other type of information structure suitable for implementing the various activities and storage functions described herein.

FIGS. 2A and 2B illustrate a front prospective view and a back prospective view, respectively, of a near-eye display device in the form of a head-mounted display (HMO) device 200 which may be implemented with an inward-facing and/or an outward-facing projection system to which examples of the present disclosure may be applied. In some examples, the head-mounted display (HMO) device 200 may be a specific implementation of the near-eye display device 120 of FIG. 1, and may be configured to operate as a virtual reality (VR) system, an augmented reality (AR) system, and/or as part of any such digital content display system that uses displays or wearables, or any combination thereof. In some examples, the head-mounted display (HMO) device 200 may include a display 210, a body 220 and a head strap 230. In some examples, the head-mounted display (HMO) device 200 may include additional, fewer, and/or different components than shown and/or described in reference to FIGS. 2A-2B.

FIG. 2A is a frontal prospective view 200A showing a front side 225, a bottom side 223, and a right side 229 of the body 220, as well as the display 210, the example outward-facing camera 250, and the head strap 230 of the head-mounted display (HMO) device 200. In some examples, two or more of the example outward-facing camera 250 may be employed for, e.g., a stereoscopic viewing by the user by display projectors inside the head-mounted display (HMO) device 200. FIG. 2B is a bottom rear prospective view 200B showing the bottom side 223, the front side 225, and a left side 227 of the body 220, as well as the display 210 and the head strap 230 of the head-mounted display (HMO) device 200. In some examples, the head strap 230 may have an adjustable or extendible length.

In particular, in some examples, there may be a sufficient space between the body 220 and the head strap 230 of the head-mounted display (HMO) device 200 for allowing a user to mount the head-mounted display (HMO) device 200 onto the user's head. For example, the length of the head strap 230 may be adjustable to accommodate a range of user head sizes.

In some examples, the head-mounted display (HMO) device 200 (including, e.g., the display 210) in FIGS. 2A-2B may include any number of processors, display electronics, and/or display optics similar to the one or more processors 121, the display electronics 122, and the display optics 124 described in reference to FIG. 1. For example, in some examples, the example outward-facing camera 250 may correspond to the outward-facing sensor(s) of the near-eye display device 120, and may be under the control of the one or more processors 121, of FIG. 1, and/or be operationally connected to any one or more of the display electronics 122, the one or more outward projectors 172, the one or more inward projectors 173, and the eye/face tracking unit 130 as indicated by the dotted line box connecting/overlapping those components in FIG. 1. The example outward-facing camera 250 in the head-mounted display (HMO) device 200 in FIGS. 2A-2B may operate similarly to the outward-facing camera(s) 320 in FIGS. 3A-3B, as discussed and described below. As mentioned above, in some examples, the head-mounted display (HMO) device 200 in FIGS. 2A-2B may include two or more outward-facing cameras rather than a single example outward-facing camera 250, such as the three outward-facing cameras employed in the Quest 3™ from Meta™.

In some examples, the display electronics and display optics of the head-mounted display (HMO) device 200 may display and/or facilitate the display of media or other digital content including virtual and/or augmented views of a physical, real-world environment with computer-generated elements. Examples of the media or digital content presented by the head-mounted display (HMO) device 200 may include images (e.g., two-dimensional (2D) or three-dimensional (3D) images), videos (e.g., 2D or 3D videos), audio, or any combination thereof. In some examples, the display electronics may display a three-dimensional (3D) image, e.g., using stereoscopic effects produced by two-dimensional panels, to create a subjective perception of image depth. In some examples, the display optics in the head-mounted display (HMO) device 200 may include a single optical element or any number of combinations of various optical elements, such as waveguides, gratings, optical lenses, optical couplers, mirrors, etc., as well as mechanical couplings to maintain relative spacing and orientation of the optical elements in the combination, such as are described above in reference to the display optics 124 in FIG. 1.

In some examples, the head-mounted display (HMO) device 200 in FIGS. 2A-2B may include one or more inward/outward projectors, similar to the one or more inward projectors 173 and/or the one or more outward projectors 172 of FIG. 1. In some examples, the one or more inward projectors of the head-mounted display (HMO) device 200 may project an image for direct observation by the user's eye and/or project a fringe or other pattern on the eye. In some examples, the one or more outward projectors of the head-mounted display (HMO) device 200 may project a fringe or other pattern on the external environment and/or objects/surfaces within the external environment in order to, for example, perform 3-dimensional (3D) mapping of the external environment. In some examples, the one or more inward/outward projectors of the head-mounted display (HMO) device 200 may include one or more of Vertical Cavity Surface Emitting Laser (VCSEL), a liquid crystal display (LCD) and/or a light-emitting diode (LED); more specifically, the one or more inward/outward projectors of the head-mounted display (HMO) device 200 may include, e.g., one or more of a liquid crystal display (LCD), a light emitting diode (LED) or micro-light emitting diode (mLED), an organic light emitting diode (OLEO), an inorganic light emitting diode (ILED), an active-matrix organic light emitting diode (AMOLED), a transparent organic light emitting diode (TLED), any other suitable light source, and/or any combination thereof. It should be appreciated that in some examples, the inward projectors of the head-mounted display (HMO) device 200 may be placed near and/or closer to a user's eye (e.g., “eye-side”). It should be appreciated that, in some instances, utilizing a back-mounted inward projector may help to reduce size or bulkiness of any required housing required for a display system, which may also result in a significant improvement in user experience for a user.

In some examples, the head-mounted display (HMO) device 200 may also include an eye/face tracking system, one or more locators, one or more position sensors, and an inertial measurement unit (IMU), similar to the eye/face tracking unit 130, the one or more locators 126, the one or more position sensors 128, and the inertial measurement unit (IMU) 132, respectively, described in reference to FIG. 1. In some examples, the head-mounted display (HMO) device 200 may include various other sensors, such as depth sensors, motion sensors, image sensors, light sensors, and/or the like. Some of these sensors may sense any number of structured or unstructured light patterns projected by the one or more inward/outward projectors of the head-mounted display (HMO) device 200 for any number of purposes, including, e.g., sensing, eye/face tracking, and/or the creation of virtual reality (VR) content.

In some examples, the head-mounted display (HMO) device 200 may include and/or be operably connected to a VR/AR/MR engine (not shown), similar to the VR/AR/MR engine 116 described in reference to FIG. 1, that may execute applications within the head-mounted display (HMO) device 200 and receive depth information, position information, acceleration information, velocity information, predicted future positions, or any combination thereof of the head-mounted display (HMO) device 200 from the various sensors. In some examples, the information received by the virtual reality engine may be used for producing a signal (e.g., display instructions) to the one or more display assemblies. In some examples, the head-mounted display (HMO) device 200 may include locators (not shown), similar to the one or more locators 126 described in reference to FIG. 1, which may be located in fixed positions on the body 220 of the head-mounted display (HMO) device 200 relative to one another and relative to a reference point. Each of the locators may emit light that is detectable by an external imaging device. This may be useful for the purposes of head tracking or other movement/orientation. It should be appreciated that other elements or components may also be used in addition or in lieu of such locators.

As stated above, the head-mounted display (HMO) device 200 may include additional, fewer, and/or different components than shown and/or described in reference to FIGS. 2A-2B. In some examples, the head-mounted display (HMO) device 200 may include an input/output interface (similar to the input/output interface 140 in FIG. 1), a console (similar to the optional console 110 described in reference to FIG. 1), and/or a camera to capture images or videos of the user's environment to present the user with, e.g., augmented reality (AR)/virtual reality (VR) content. In some examples, the head-mounted display (HMO) device 200 may include one or more cameras to capture reflections of patterns projected by the one or more inward/outward projectors.

FIGS. 3A and 3B illustrate a perspective view 300A and a top view 300B, respectively, of a near-eye display device 300 in the form of a pair of glasses having both an inward-facing and an outward-facing projection systems to which examples of the present disclosure may be applied. In some examples, the near-eye display device 300 may be a specific implementation of the near-eye display device 120 of FIG. 1, and may be configured to operate as a virtual reality (VR) system, an augmented reality (AR) system, and/or as part of any such content system that uses displays or wearables, or any combination thereof. As shown in FIGS. 3A-3B, the near-eye display device 300 may include a frame 305, one or more outward pattern projectors 310, one or more eye/face tracking projectors 315 (which effectively operate as inward pattern projectors), an outward-facing camera(s) 320, an eye/face tracking camera(s) 325, and a display 390.

As shown in FIGS. 3A-3B, the near-eye display device 300 may include an inward-facing imaging/projection system, which may include the one or more eye/face tracking projectors 315 (i.e., inward pattern projectors) and the eye/face tracking camera(s) 325, and an outward-facing imaging/projection system, which may include the one or more outward pattern projectors 310 and the outward-facing camera(s) 320. In some examples, the inward-facing imaging/projection system of the near-eye display device 300 may be an eye/face tracking system, where the one or more eye/face tracking projectors 315 project a pattern directly on the user's eye(s) and/or face, and the eye/face tracking camera(s) 325 captures one or more reflections of the projected pattern from the user's eye(s) and/or face, and the eye/face tracking system uses the captured reflections to track the user's eye(s) and/or face. In some examples, the one or more outward pattern projectors 310 may include, e.g., infrared (IR) projectors, and the one or more outward-facing camera(s) 320 may include one or more cameras which are part of a Simultaneous Localization and Mapping (SLAM) system for tracking the position and orientation of the near-eye display device 300 in real-time and/or mapping the external environment in 3D.

In some examples, the one or more eye/face tracking projectors 315 may be disposed on the temple arms of the frame 305 of the near-eye display device 300 (not shown in either FIG. 3A or 3B), and may project one or more patterns on eye lens of the near-eye display device 300, which reflects those one or more patterns onto the user's eye 355 and/or face (i.e., a rear projection slight source). In such examples, the inner surface of the eye lens may be coated with a reflective surface, fabricated with a reflective surface, and/or covered by a metasurface or other type of nanostructure which may be suitably employed for the re-direction of the light projected by the one or more eye/face tracking projectors 315, as would be understood by one of ordinary skill in the art. In such examples, the inner surface may create the one or more patterns which are projected onto the user's eye 355 and/or face, either alone or in combination with the one or more eye/face tracking projectors 315. In other words, in some examples, the one or more eye/face tracking projectors 315 may project unstructured light, and the inner surface both reflects and/or re-directs the light onto the user's eye and/or face while also providing one or more patterns which may be used for eye/face tracking. In some examples, the one or more eye/face tracking projectors 315 may project a pattern such as, for example, a structured image (e.g., a fringe pattern) projected onto the eye and/or face by a micro-electromechanical system (MEMS) based scanner reflecting light from a light source (e.g., a laser).

In some examples, the one or more eye/face tracking projectors 315 may include one or more of a light emitting diode (LED) or micro-light emitting diode (mLED) or edge emitting LED, an organic light emitting diode (OLEO), an inorganic light emitting diode (ILED), an active-matrix organic light emitting diode (AMOLED), a transparent organic light emitting diode (TLED), a superluminiscent diode (SLED), another type of suitable light emitting diode, a Vertical Cavity Surface Emitting Laser (VCSEL) or other type of laser, a photonic integrated circuit (PIC) based illuminator, a liquid crystal display (LCD), a light source with a micro-electromechanical system (MEMS) based scanner, any other suitable light source, and/or any combination thereof.

In any examples employing a VCSEL, the VCSEL may have one or more of a wide variety of possible VCSEL architectures, and/or fabrications, as would be understood by one of ordinary skill in the art. In such examples, the VCSEL may include a VCSEL with multiple active regions (e.g., a bipolar cascade VCSEL); a tunnel junction VCSEL; a tunable VCSEL which may employ, e.g., a micro-electromechanical system (MEMS); a wafer-bonded and/or wafer-fused VCSEL; a Vertical External Cavity Surface Emitting Laser (VECSEL); a Vertical Cavity Semiconductor Optical Amplifier (VCSOA) which may be optimized as amplifiers as opposed to oscillators; two or more Vertical Cavity Surface Emitting Lasers (VCSELs) disposed on top of one another (i.e., vertically) such that each one pumps the one on top of it (e.g., monolithically optically pumped VCSELs); any other suitable VCSEL construction, architecture, and/or fabrication, as would be understood by one of ordinary skill in the art in light of the examples of the present disclosure; and/or other constructions, architectures, and/or fabrications suitable for the present disclosure may be employed besides a VCSEL, such as-with appropriate architectural modifications, for example, an Edge-Emitting Laser (EEL), a Horizontal Cavity Surface Emitting Laser (HC-SEL), a Quantum Dot Laser (QDL), a Quantum Cascade Laser (QCL), any other form of solid state laser, and/or any light source suitable for examples according to the present disclosure, as would also be understood by one of ordinary skill in the art.

In some examples, the eye/face tracking camera(s) 325 may be an image sensor, such as a complementary metal-oxide semiconductor (CMOS) image sensor, a defocused image sensor, a light field sensor, a single photon avalanche diode (SPAD), and/or, in certain implementations, a non-imaging sensor, such as a self-mixing interferometer (SMI) sensor. In some examples, a combined VCSEL/SMI integrated circuit may be employed as both a light source and a sensor for eye/face tracking. In such an example employing a combined VCSEL/SMI integrated circuit as both a light source and a sensor for eye/face tracking, the combined VCSEL/SMI integrated circuit may be disposed inside the frame of near-eye display device 300 and point into the waveguide 393 constituting the display 390 in order to perform eye/face tracking illumination and sensing.

As shown in FIG. 3B, in some examples, the outward-facing imaging/projection system of the near-eye display device 300 may include the one or more outward pattern projectors 310, which project a pattern directly on an external environment 350 and/or one or more objects/surfaces in the external environment 350, and the outward-facing camera(s) 320, which captures one or more reflections of the projected pattern on the one or more objects/surfaces or all or part of the external environment 350. In some examples, such an outward-facing imaging/projection system may serve a variety of purposes, including, but not limited to, profilometry, determining surface patterns/structures of objects in the external environment 350, determining distances from the user to one or more objects/surfaces in the external environment 350, determining relative positions of one or more objects/surfaces to each other in the external environment 350, determining relative velocities of one or more objects/surfaces in the external environment 350, etc., as would be understood by one of ordinary skill in the art. In some examples, the outward-facing imaging/projection system of the near-eye display device 300 may also be employed to capture images of the external environment 350. In such examples, the captured images may be processed, for example, by a virtual reality engine to add virtual objects to the captured images or modify physical objects in the captured images, and the processed images may be displayed to the user by the display 390 for augmented reality (AR) applications.

In some examples, the display 390 may include, in whole or in part, one or more processors, display electronics, and/or display optics similar to the one or more processors 121, the display electronics 122, and the display optics 124 in FIG. 1, and may be configured to present media or other content to a user, including, e.g., virtual reality (VR), augmented reality (AR) system, and/or any other system capable of presenting media or other content to a user. In some examples, the display 390 may include any number of light sources, such as, e.g., Vertical Cavity Surface Emitting Laser (VCSEL), a liquid crystal display (LCD) display panel, a light-emitting diode (LED) display panel, or an optical display panel (e.g., a waveguide display assembly), etc., and any number of optical components, such as waveguides, gratings, lenses, mirrors, etc., as would be understood by one of ordinary skill in the art.

As shown in FIG. 3B, in some examples, the display 390 of the near-eye display device 300 may include optics 391 and a waveguide 393, which may be coupled to a projector (such as, e.g., the one or more inward projectors 173 of FIG. 1). In some examples, the projectors may be disposed inside the frame on the sides of the waveguide 393 constituting the display 390, thereby projecting light into and through the waveguide 393, which, in turn, projects the light towards the user's eye. In some examples, the display 390 may combine the view of the external environment 350 and artificial reality content (e.g., computer-generated images). In some examples, light from the external environment 350 may traverse a “see-through” region of the waveguide 393 in the display 390 to reach a user's eye 355 (located somewhere within an eye box), while images are also projected for the user to see as part of an augmented reality (AR) display.

In such examples, the light of images projected by the projector may be coupled into a transparent substrate of the waveguide 393, propagate within the waveguide 393, be coupled with light from the user's actual environment, and be directed out of the waveguide 393 at one or more locations towards a user's eye 355 located within the eye box. In such examples, the waveguide 393 may be geometric, reflective, refractive, polarized, diffractive, and/or holographic, as would be understood of one of ordinary skill in the art, and may use any one or more of macro-optics, micro-optics, and/or nano optics (such as, e.g., metalenses and/or metasurfaces). In some examples, the optics 391 of the display 390 may include optical polymers, plastic, glass, transparent wafers (e.g., Silicon Carbide (SIC) wafers), amorphous silicon, Silicon Oxide (SiO₂), Silicon Nitride (SIN), Titanium Oxide (TiO), optical nylon, carbon-polymers, and/or any other transparent materials used for such a purpose, as would be understood by one of ordinary skill in the art.

In some examples, the near-eye display device 300 may further include various sensors on or within a frame 305, such as, e.g., any number of depth sensors, motion sensors, position sensors, inertial sensors, and/or ambient light sensors. In some examples, the various sensors may include any number of image sensors configured to generate image data representing different fields of views in one or more different directions (which may or may not include the outward-facing camera(s) 320). In some examples, the various sensors may be used as input devices to control or influence the displayed content of the near-eye display device 300, and/or to provide an interactive virtual reality (VR) and/or augmented reality (AR) experience to a user of the near-eye display device 300. In some examples, the various sensors may also be used for stereoscopic imaging or other similar application.

In some examples, the near-eye display device 300 may further include one or more illuminators to project light into a physical environment (which may or may not include, e.g., the outward pattern projector(s) 310). The projected light may be associated with different frequency bands (e.g., visible light, infra-red light, ultra-violet light, etc.), and may serve various purposes. In some examples, the one or more illuminators may be used as locators, such as the one or more locators 126 described above with respect to FIG. 1. In such examples, the near-eye display device 300 may also include an image capture unit (which may or may not include the outward-facing camera(s) 320 and/or the external imaging device), which may capture images of the physical environment in the field of view. In some instances, the captured images may be processed, for example, by a VR/AR/MR engine (such as, e.g., the VR/AR/MR engine 116 of FIG. 1) to add virtual objects to the captured images or modify physical objects in the captured images, and the processed images may be displayed to the user by the display 390 for augmented reality (AR) applications.

In some examples, a majority of electronic components of the near-eye display device 300 in the form of a pair of glasses may be included in the frame 305 of the glasses (e.g., a top bar, a bridge, a rim, a lens, etc.). Examples of such electronic components included in the frame 305 include, but are not limited to, a camera, a sensor, a projector, a speaker, a battery, a microphone, and a battery management unit (BMU). In some examples, a battery management unit (BMU) may be an electronic system that may be used to manage charging and discharging of a battery (e.g., a lead acid battery). In some examples, the battery management unit (BMU) may, among other things, monitor a state of the battery, determine and report data associated with the battery, and provide environmental control(s) for the battery. In some examples, the temples may be provided with a tapering profile, based on design considerations for the specific implementation. In such examples, the tapered temples may be utilized to house various electronic components. For example, in some cases, a microphone or speaker may often be placed towards a rear of a temple arm, near a user's ear, and as such, in many cases, a battery may be more likely to be placed near a front of the temple arm.

In FIG. 3B, an eye/face tracking system (such as that described in reference to eye/face tracking unit 130, the eye/face tracking module 118, and the one or more inward projector(s) 173 of FIG. 1) may be implemented by the eye/face tracking projector(s) 315, which project structured light, such as patterns and/or other suitable lighting for performing eye/face tracking upon the user's eye 355 and/or portions of the user's face, the eye/face tracking camera(s) 325, which receive reflections of the light of the eye/face tracking projector(s) 315 from the user's eye 355 and/or portions of the user's face, and a controller (or controllers) 317, which process the reflections received by the eye/face tracking camera(s) 325 to perform eye/face tracking. In some examples, the structured light may include one or more patterns. In some examples, the projected structured light may include, for example, one or more of a statistically random pattern (such as, e.g., a pattern of dots or a pattern of speckles), an interference pattern (such as, e.g., a moire pattern or a fringe pattern), a sinusoidal pattern, a binary pattern, a multi-level pattern (such as, e.g., a multi-level grayscale pattern), a code-based pattern, a color-based pattern, and a geometrical pattern (such as, e.g., a triangular, pyramidal, or trapezoidal pattern), as would be understood by one of ordinary skill in the art. Additionally, in various examples of the present disclosure, there may be only one projected pattern, or a multitude of patterns, or a series of related patterns, which may be projected either separately, in a time series, or simultaneously, as would be understood by one of ordinary skill in the art. In some examples, periodic patterns (such as, e.g., fringe patterns) and/or non-periodic patterns (such as, e.g., speckle patterns) may be employed.

In some examples, the controller 317 may be similar to the one or more processors 121 in FIG. 1 (and thus may perform a wide variety of functions for the near-eye display device 300), other processors which perform several tasks, and/or a processors dedicated to performing eye tracking and/or face tracking. In some examples, the controller 317 for performing eye tracking and/or face tracking may be communicatively connected with a memory, which may be a non-transitory computer-readable storage medium storing instructions executable by the controller 317. The controller 317 may include multiple processing units, and those multiple processing units may further execute instructions in parallel. The non-transitory computer-readable storage medium may be any memory, such as a hard disk drive, a removable memory, or a solid-state drive (e.g., flash memory or dynamic random access memory (DRAM)). In various examples, the controller 317 may be further subdivided into multiple devices (for example, the functions of the controller 317 may be separated among various components, such as a digital signal processing (DSP) chip for eye and/or face tracking analysis as well as a Central Processing Unit (CPU) for controlling, e.g., the eye/face tracking projector(s) 315).

As mentioned above, it may be desirable to incorporate health monitoring into wearable devices and, more particularly, to be capable of heart health measurements in a near-eye device, such as, for example, a near-eye VR/AR/MR display device. Besides health and fitness monitoring, tracking, etc., such a heart health measurement capability may indicate the user's emotional and physiological state, which may be used for a variety of purposes, such as, in the example of a near-eye VR/AR/MR display device, enabling more personalized and immersive interactions within the VR/AR/MR environment. However, there may be limitations in terms of space, power, and other resources/capabilities of a near-eye device when attempting to add a sensor system for monitoring heart health.

In near-eye devices according to the present disclosure, the pre-existing eye/face tracking system may be suitably employed to sense changes in the vasculature of the eye and/or the surrounding facial tissue in order to measure, monitor, and/or track heart health (such as, e.g., the heart rate). In some examples, the eye/face tracking system may be similar to any one or more of the eye/face tracking unit/module 130/118 in FIG. 1, an eye/face tracking system in the head-mounted display (HMO) device (HMO) 200 of FIGS. 2A-2B, and/or the eye/face tracking cameras/projectors 325/325 (and possibly the controller 317) in the near-eye display device 300 of FIGS. 3A-3B; in other examples, the eye/face tracking system may include other type of near-eye devices.

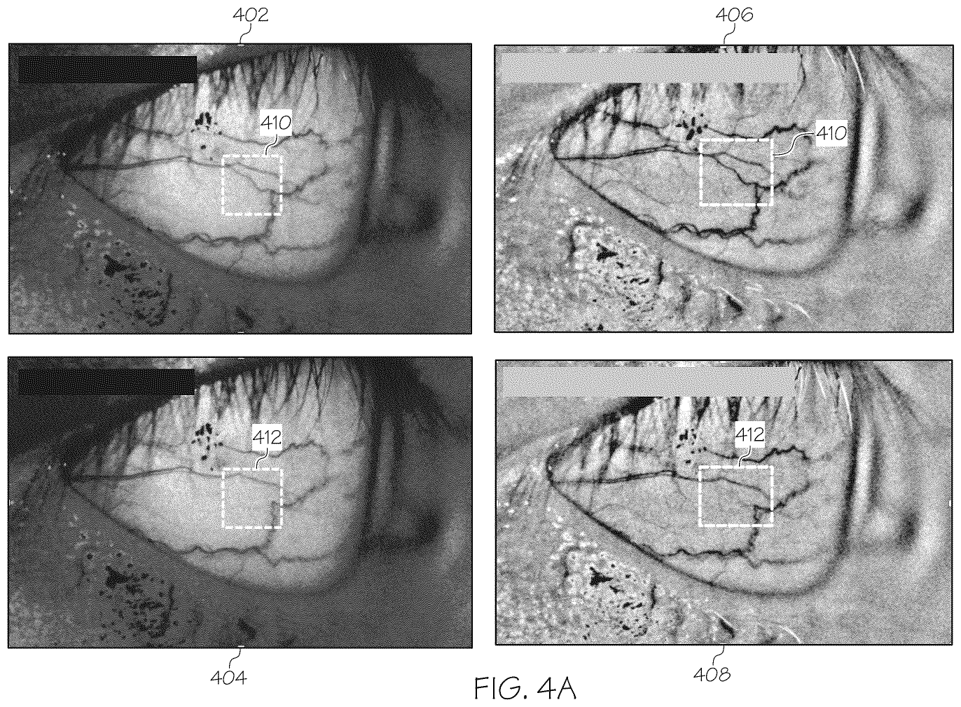

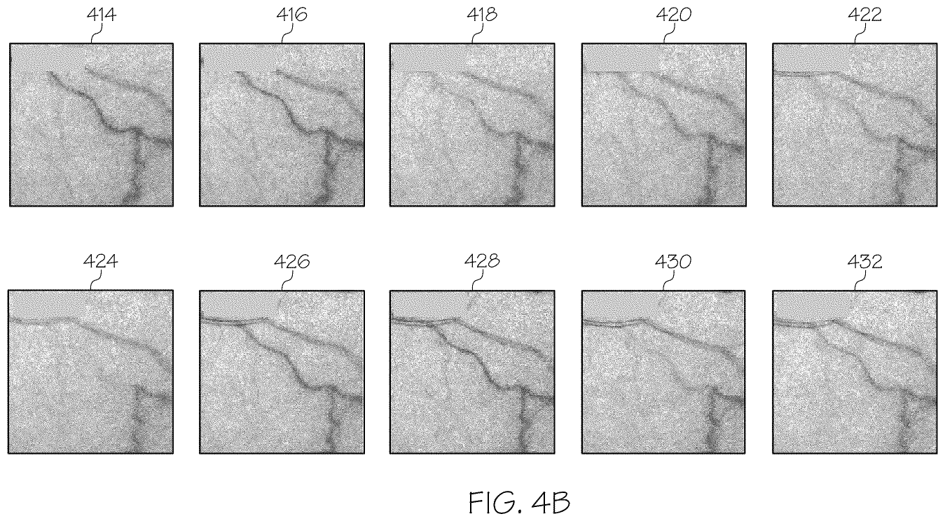

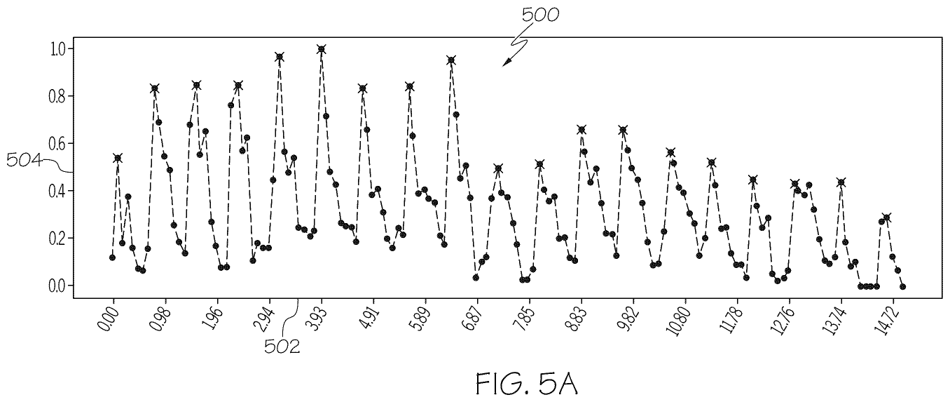

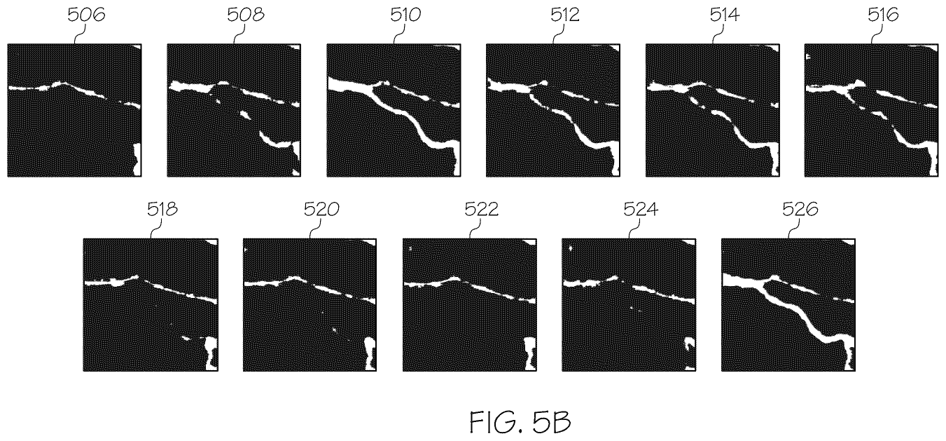

In some examples, a coherent light source (such as, e.g., laser diode(s), an array of Vertical Cavity Surface Emitting Lasers (VCSELs), etc.) of an eye/face tracking system in a near-eye device may be employed to both illuminate and image the clear conjunctival and episcleral vasculatures of the eye sclera. In some examples, speckle contrast may be utilized to observe the blood flow pulses in the eye vasculature as periodic intensity changes in the blood vessels. Such periodic intensity changes may be employed to quantify, observe, and/or monitor the heart rate of the user. Speckle contrast techniques, such as Laser Speckle Contrast Imaging (LSCI) and Laser Speckle Imaging (LSI, are discussed in further detail below, for example, in reference to FIGS. 4A-4B and 5A-5B.

In some examples, the coherent illumination and detection components that are already part of the eye/face tracking system off a near-eye device may be employed to capture cardiac dynamics, such as, e.g., changes in heart rate. The derivative metrics of the vasculature of the eye and/or (possibly) surrounding facial tissue, such as, e.g., the change in width and distribution of capillaries over time, may be utilized to determine whether possible diagnostic criteria are met, such as, for example, elevated eye pressure and/or changes in pressure over time may indicate a risk for stroke and/or a trend for concern in those congenitally and/or physiologically predisposed for stroke. Depending on the sensitivity of the system and predictability of the diagnostic criteria, it may be contemplated that a near-eye device with an eye/face tracking system capable of health monitoring in accordance with the present disclosure may be able to recognize that a stroke is occurring in the user.

FIGS. 4A-4B and 5A-5B provide examples and additional information regarding using speckle contrast imaging for monitoring cardiac dynamics. More specifically, examples and additional information regarding Laser Speckle Contrast Imaging (LSCI) and/or Laser Speckle Imaging (LSI) are described and discussed. In LSCI or LSI, a laser projects a speckle pattern on an object or surface and the reflections of that speckle pattern are imaged and analyzed to, for example, detect motion where there is an otherwise motionless background. Specifically, in a temporal series of images (or frames), the pixel areas which fluctuate, or are attenuated, or blurred, are the specific areas where movement has occurred. Because the superficial retinal tissue of the eye is a highly scattering medium, over time periods where the eye is not moving, the background tissue, which is not moving, produces a constant speckle pattern, while the blood vessels or capillaries near the surface generate temporally varying speckle patterns due to the flow of scattering particles—i.e., the red blood cells flowing through the capillaries. Speckle statistics may be calculated using the neighboring (background) pixels in comparison with the blurred/moving (capillary) pixels to both create blood vessel/capillary maps of the eye and determine relative flow magnitudes-either or both of which may be employed to monitor heart health characteristics such as, for example, heart rate. For theoretical background and general information, see, e.g., A Fercher & D.

FIGS. 4A-4B illustrate eye vasculature visualization using speckle contrast, which may be utilized in accordance with examples of the present disclosure. As stated in FIG. 4A, speckle contrast quantifies the blurriness of speckle patterns caused by the rapid motion of scatterers (e.g., blood cells) during the camera exposure time. Eye vasculatures (i.e., conjunctival and episcleral vasculatures) may be visible in speckle contrast images. The images (examples images 402, 404, 406, and 408) in FIG. 4A are based upon a 163 fps raw video, where the speckle contrast is calculated using a 10-frame moving window. The example images two images on the top show (example images 402 and 406) the eye at roughly time point 0.07.43 of the video, where the capillary in the center of the white dashed box is fairly full of blood (example blood-filled capillary 410), while the two images on the bottom (example images 404 and 408) show the eye at roughly time point 0.03.07 of the video, where the capillary in the center of the white dashed box is roughly empty of blood (exampled bloodless capillary 412) and thus mostly unseen. Accordingly, it may be demonstrated that speckle contrast imaging may capture the pulsing of the heart, i.e., the heart rate.

As shown in FIG. 4B, the blood flow pulses in eye capillaries may be observed in speckle contrast images as periodic intensity changes in blood vessels. More specifically, the ten images of FIG. 4B (examples images 414, 416, 418, 420, 422, 424, 426, 428, 430, and 432) are a time series of frames of an area roughly equivalent to the white dashed box area shown in FIG. 4A. These images depict the eye capillary pulsing and changing over a particular time frame, e.g., from 0.000 s, which is associated with example image 414, to 0.883 s, which is associated with the example image 432. In aspects, each image is obtained at roughly 0.1 s apart intervals. At 0.000 s image/frame, the indicated eye capillary is full, whereas at, e.g., 0.491 s, the eye capillary may be almost completely invisible, thus indicating that it is empty (example image 424).

FIGS. 5A-5B illustrate the measurement of heart rate based on blood flow pulses in eye capillaries, which may be employed in accordance with examples of the present disclosure. FIG. 5A is a graph 500 that has an x-axis 502 that corresponds to time and a y-axis 504 that corresponds to pulse values (normative value). Further, the graph 500 includes markers in the form of “x” and “dots” where the “x” markers represent peak values and the dots represent images taken at roughly 1/10 of a second intervals. The graph 500 has an average pulse rate of around 74.34 bpm. As indicated in FIG. 5B, a pilot study was performed comparing readings from speckle contrast imaging with those obtained using a smartwatch. The smartwatch readings were obtained using the technique of Photophlethysmography. Examples images 506, 508, 510, 512, 514, 516, 518, 520, 522, 524, and 526 depict heart rate readings obtained using speckle imaging. The readings obtained from speckle imaging were more accurate (e.g., 74.34 beats per minute) than those obtained using the smartwatch (approximately 76 beats per minute).

Generally speaking, monitoring by speckle imaging using the eye/face tracking systems of near-eye devices may include a number of different use cases and applications, e.g., health monitoring, emotion detection, user experience enhancement, safety, and research and training. Specifically, continuous heart monitoring may provide valuable insights into the user's health and fitness. This may be particularly useful information for wellness/fitness applications, e.g., allowing the user to track their heart rate during exercise or meditation sessions. Additionally, changes in heart rate may indicate emotional responses such as excitement, fear, a relaxed state, etc., which can be used in VR/AR/MR applications to gauge a user's reaction to interactive content and adapt the content accordingly. For instance, a horror VR game or other such VR/AR/MR content may actively change itself, in terms of, e.g., “scare factor,” if the heart rate indicates the user is not frightened. Heart rate data can also be utilized to personalize and adapt a VR/AR/MR experience in near-real-time. For instance, in a VR game or other such VR/AR/MR content, the intensity or difficulty level may be adjusted based on the user's heart rate, creating a more immersive and engaging experience. In another instance, monitoring the user's heart rate may help ensure their safety. For example, if the user's heart rate becomes dangerously high in a high-intensity VR game or other such VR/AR/MR content, the system could alert the user and/or automatically adjust the intensity of the high-intensity VR game (or other such content). In yet another instance, heart rate data may be used for research and/or training purposes such as in medical and/or military training simulations. As part of these simulations, the trainees' heart rates may provide valuable feedback on their stress levels and/or performance under pressure.

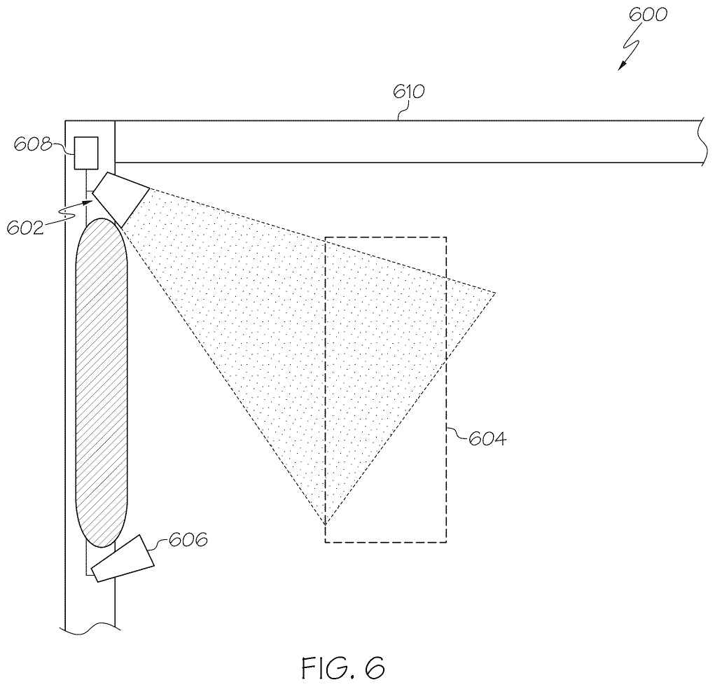

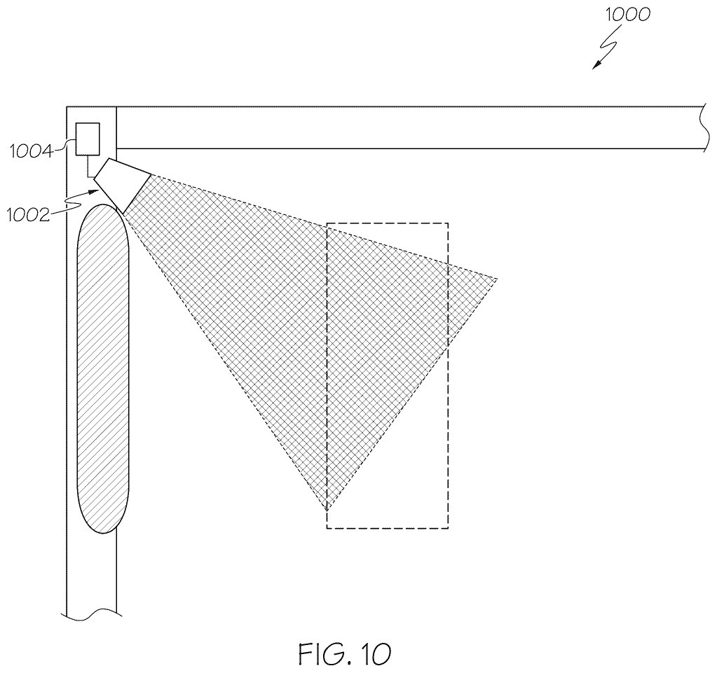

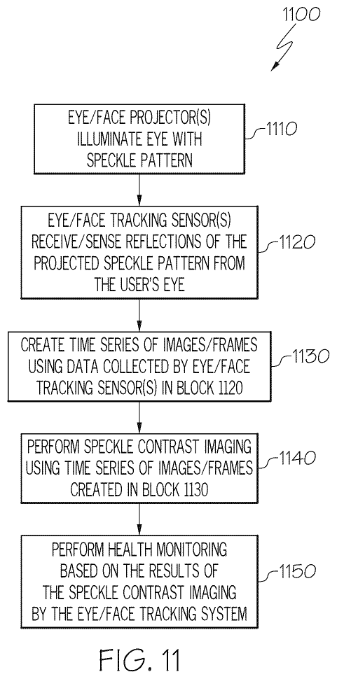

Below, non-limiting examples of eye/face tracking systems of near-eye devices being utilized to perform speckle contrast imaging for health monitoring are described in reference to FIGS. 6-9B; a non-limiting example of a high resolution eye/face tracking camera of a near-eye device being utilized to detect heart beat microtremors for health monitoring is described in reference to FIG. 10; and non-limiting examples of methods for health monitoring using speckle contrast imaging performed, at least in part, by an eye/face tracking system of a near-eye device are described in reference to FIGS. 11-13C.

FIG. 6 is a block diagram of an eye-face tracking system 600 in a near-eye device, with a global shutter eye-face tracking camera 602 for health monitoring, according to an example of the present disclosure. FIG. 6 is provided to illustrate a general explanation herein of examples of an eye/face tracking system 600 in a near-eye device, with a global shutter eye-face tracking camera 602 for health monitoring, and omits aspects, features, and/or components not germane to a general explanation of examples, with a global shutter eye-face tracking camera 602 for health monitoring, according to the present disclosure, as would be understood by one of ordinary skill in the art. Accordingly, the components shown in FIG. 6 may not be presented in accurate aspect and/or ratio of relative sizes (e.g., the relative sizes, shapes, and/or locations of the eye/face tracking camera(s), the eye/face tracking projector(s), the eye lens, the frame of the near-eye device, etc. Further, FIG. 6 may not approximate the sizes relative locations, and/or relative dimensions of those components in specific implementations and/or examples). In other words, FIG. 6 is intended to illustrate general concepts related to examples of the present disclosure, and is not intended to illustrate the sizes, proportions, relative aspects, etc., of the specific components shown in FIG. 6, as would be understood by one of ordinary skill in the art.

FIG. 6 shows a partial cross-section of the frame of a near-eye device, within which an eye lens may be disposed such that an eye in the eye box 604 (the gray dashed line box) may look into it, while both the global shutter eye/face tracking camera 602 and an eye/face tracking coherent light source 606 are pointing towards the eye box 604. The eye/face tracking coherent light source 606 illuminates the eye box with speckle contrast imaging, and the global shutter eye/face tracking camera 602 receives reflections from the eye of the speckle contrast images. An eye/face tracking controller 608 may be operatively connected to, and control, both the eye/face tracking coherent light source 606 and the global shutter eye/face tracking camera 602 in order to perform speckle contrast imaging, such as, e.g., LSCI/LSI.

In some examples, a partial frame section 610 as in FIG. 6 may be a section of the front portion of the frame 305 of the near-eye display device 300 in FIGS. 3A-3B. In other examples, the partial frame section 610 may be a part of the front side 225 of the HMO in FIGS. 2A-2B; in yet other examples, the partial frame section 610 may be included in a near-eye device with a completely different shape and appearance. In some examples, the global shutter eye/face tracking camera 602 may be the eye/face tracking camera(s) 325 of FIGS. 3A-3B, and/or part of the eye/face tracking unit 130 and/or the eye/face tracking module 118 of FIG. 1. In some examples, the eye/face tracking coherent light source 606 may be the one or more inward projector(s) 172 of FIG. 1 and/or the eye/face tracking projector(s) 315 of FIGS. 3A-3B.

In some examples, the eye/face tracking controller 608 may constitute multiple components involved with performing eye/face tracking for the near-eye device. In some examples, the eye/face tracking controller 608 may be the eye/face tracking unit 130 and/or the eye/face tracking module 118 of FIG. 1, or any other such eye/face tracking processing system. In some examples, the eye/face tracking controller 608 may include a processor (not shown) and/or a memory (not shown), which may be a non-transitory computer-readable storage medium (and may store instructions executable by the processor and/or the eye/face tracking controller). In some examples, the eye/face tracking controller 608 may perform any of the methods, functions, and/or processes described in any portion of the present disclosure by executing instructions contained on any suitable non-transitory computer-readable storage medium. In other examples, one or more other processors besides, or in addition to, the eye/face tracking controller, may perform any of the methods, functions, and/or processes described in any portion of the present disclosure by executing instructions contained on any suitable non-transitory computer-readable storage medium.

In examples employing Laser Speckle Contrast Imaging (LSCI) or Laser Speckle Imaging (LSI), the eye/face tracking projector projects a speckle pattern towards the eye box 604 and reflections of that speckle pattern are imaged by the global shutter eye/face tracking camera 602 and analyzed by, for example, the eye/face tracking controller 608 to, e.g., detect motion where there is an otherwise motionless background. For instance, as shown in FIGS. 4A-4B and 5A-5B, the pixel areas which fluctuate, are attenuated, blurred, and/or otherwise vary over time in a temporal series of images (or frames) are the specific areas where movement has occurred. Accordingly, because the superficial retinal tissue of the eye is a highly scattering medium, over time periods where the eye is not moving, the background tissue, which is not moving, produces a constant speckle pattern, while the blood vessels or capillaries near the surface generate temporally varying speckle patterns due to the flow of scattering particles—i.e., the red blood cells flowing through the capillaries. Speckle statistics may be calculated using the neighboring (background) pixels in comparison with the blurred/moving (capillary) pixels to both create blood vessel/capillary maps of the eye and determine relative flow magnitudes-either or both of which may be used to monitor cardiac characteristics, such as, e.g., heart rate, pulse rhythm, blood pressure, etc.

Thus, in examples in accordance with the present disclosure, during sufficiently long enough time periods when the eye is stationary (i.e., motionless, still, not currently moving), data may be collected from the eye tracking camera(s) to form a time-series sequence of frame/images. In some examples, a sufficiently long enough time period may be less than a second, when a few dozen to a few hundred frames/images may be taken/obtained. Speckle contrast (which is a function of the exposure time of the camera and is related to the autocovariance of the intensity fluctuations in individual speckles), or any other suitable descriptor of temporal speckle statistics, is computed over the time series sequence of frame/images, whereby, for example, the eye/face tracking controller (and/or other suitable processor) may, e.g., extract the location of the sub-surface blood vessels (e.g., capillaries) as well as the velocity of the blood flow through those blood vessels. In such examples, the eye/face tracking controller may determine a map of the surface capillaries of the eye and/or the blood flow dynamics or hemodynamics of those capillaries, including, e.g., changes in blood flow within the capillaries; the viscosity of the blood plasma; the shapes and dynamics of the red blood cells; the osmotic pressure within the capillaries; hemodilution; the turbulence and velocity of blood flow; vascular resistance, stress, capacitance, and/or wall tension; etc., all of which measurements/criteria/diagnostic tools would be known and understood by one of ordinary skill in the art. In such a manner, examples in accordance with the present disclosure may detect, measure/quantify, monitor, etc., features from which the user's health may be directly determined and/or indirectly inferred/calculated based on pattern recognition/machine learning (ML), etc. In some examples, data acquisition during the pupil's stationary state may last several data acquisition frames. Depending on a sensing frame rate, the actual stationary state may last 10-100 milliseconds.

In some examples, a few dozen to a few hundred frames/images in a single second may be used to perform such processing. In some examples, the frames/images may not all need to be in sequence to perform the user authentication and liveness detection in accordance with the present disclosure. In some examples, out-of-sequence frames may be preferred as the sensing method but may also be impacted by speckle de-correlation time. In some examples, speckle de-correlation time may be affected by the fact that, in order for there to be a noticeable change in speckle pattern, there should be sufficient movement, motion, and/or other form of physical change (such as, e.g., the blood cells move sufficiently between images/frames). Alignment of the images (to landmarks, such as pupil, iris corners of the eye) is sufficient for performance of statistical analysis on speckle patterns at same physical locations. Other techniques for data processing may also be employed, as would be understood by one of ordinary skill in the art.

In FIG. 6, the global shutter eye/face tracking camera 602 may operate in a global fashion, i.e., each frame/image is taken of all and/or most pixels at the same time. Accordingly, the eye/face tracking controller 608 (and/or other processor(s)) may process an entire frame/image at a time when performing speckle contrast imaging, or, at the least, receive (or have accessible) the pixel data of an entire image/frame at substantially the same clock time.

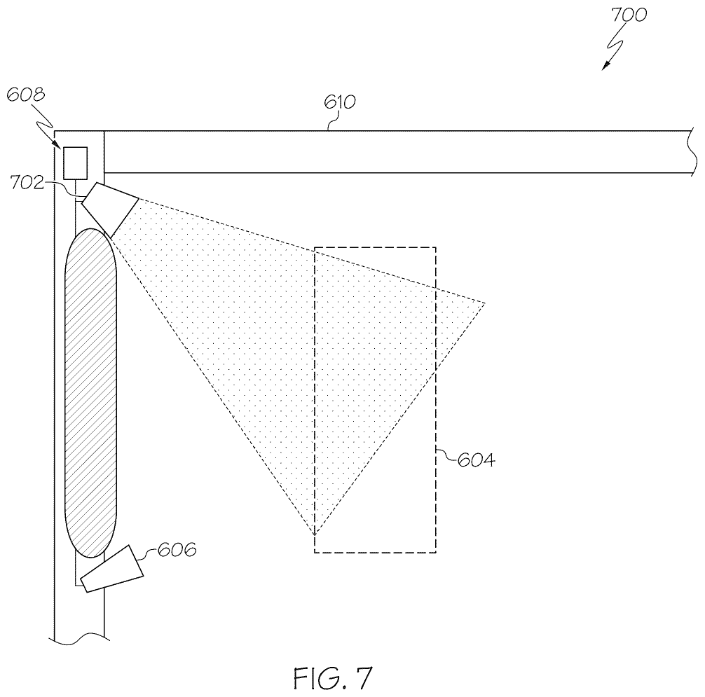

FIG. 7 is a block diagram of another eye-face tracking system 700 in a near-eye device, with a rolling shutter eye-face tracking camera 702 for health monitoring, according to an example of the present disclosure. All of the description above concerning FIG. 6 applies equally to FIG. 7, except that, instead of the global shutter eye/face tracking camera 602 in FIG. 6, FIG. 7 has the rolling shutter eye/face tracking camera 702. In FIG. 7, the rolling shutter eye/face tracking camera 702 may operate in a rolling shutter fashion, e.g., instead of all or most of the pixels of each frame/image being taken at the same time, only a portion, such as a line of pixels, are taken at the same time (e.g., “line-by-line”). Accordingly, only portions of the entire frame/image are available/accessible at a time to the eye/face tracking controller (and/or other processor(s)) when performing speckle contrast imaging. However, in such implementations/examples, more simple, cost-effective, faster, and/or less resource-intensive eye/face tracking camera(s) and/or eye/face tracking controller(s) (and/or other processor(s) employed for the sparkle contrast imaging) may be employed.

Rolling shutter cameras (e.g., the rolling shutter camera 702) may offer several technical advantages over global shutter cameras in motion tracking applications. Rolling shutter cameras are generally more cost-effective and thus more practical due to their less complex circuitry. In addition, rolling shutter cameras may provide higher resolution, resulting in more detailed and accurate motion tracking. Furthermore, the rolling shutter camera 702 may provide superior performance in low light conditions, because each pixel has more time to gather light, enhancing image quality in poorly lit environments.

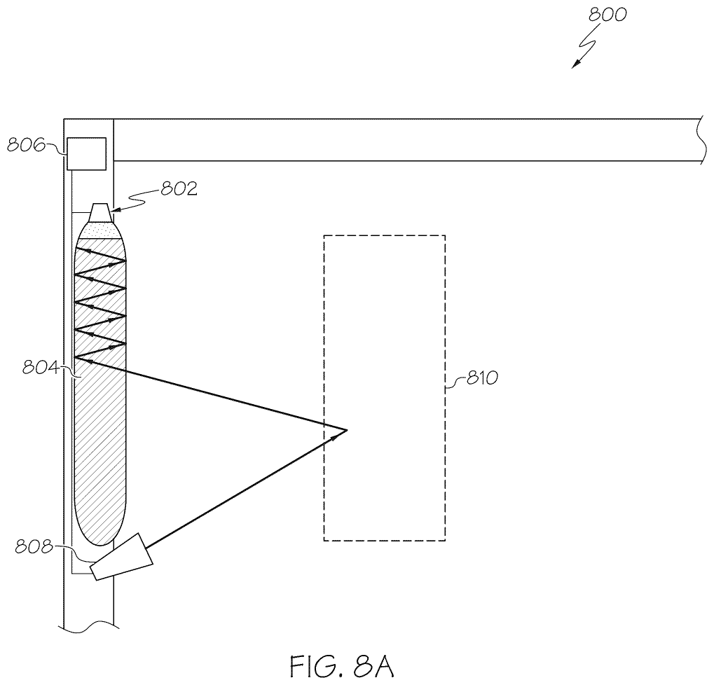

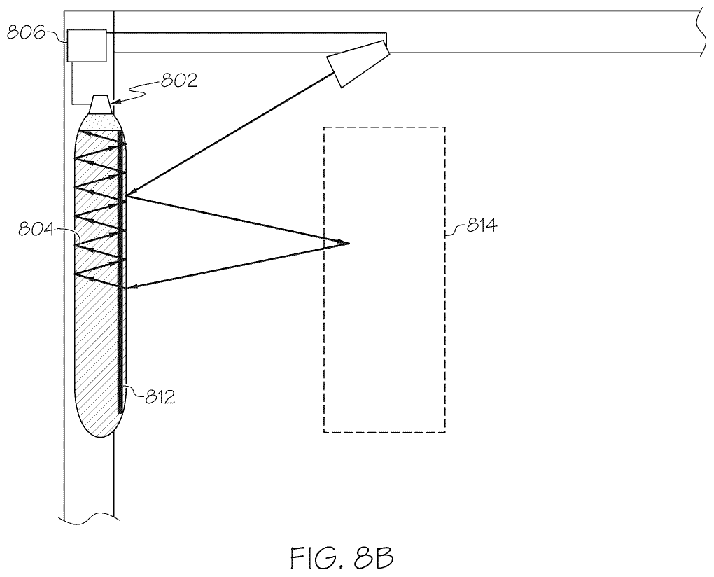

FIGS. 8A-8B are block diagrams of an example eye-face tracking system 800 in a near-eye device, in which an example eye-face tracking camera 802 (e.g., either the global shutter eye-face tracking camera 602 or the rolling shutter eye-face tracking camera 702) can be pointed into an example waveguide 804 and is capable of health monitoring, according to examples of the present disclosure. All descriptions above concerning FIGS. 6 and 7 apply equally to FIGS. 8A-8B, except that (1) the example eye/face tracking camera 802 in FIGS. 8A-8B may be either the global shutter eye/face tracking camera 602 as in FIG. 6 or the rolling shutter eye/face tracking camera 702 as in FIG. 7; (2) an eye lens structure can be an example waveguide 804 (e.g., the waveguide 393) through which light may propagate by internal reflection (as discussed above in reference to, e.g., FIGS. 3A-3B); (3) an example eye/face tracking controller 806 in FIGS. 8A-8B receives reflections from the user's eye through the waveguide 804 (in light blue); and (4) an example eye/face tracking projector 808 may be located either in the front part of the frame of the near-eye device facing an example eye box 810 (i.e., FIG. 8A) or on the temple of the near-eye device.

Further, in aspects, as shown in FIG. 8B, the example waveguide 804 can include a hot mirror 812 disposed therein. The hot mirror 812 can be utilized to reflect the projected light into an example eye box 814. In aspects, infrared (IR) and/or near-IR (NIR) light can reflect from the hot mirror, but all or most light in the visible spectrum may pass through the hot mirror.

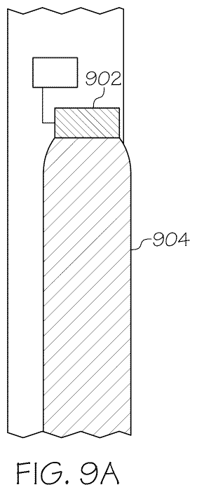

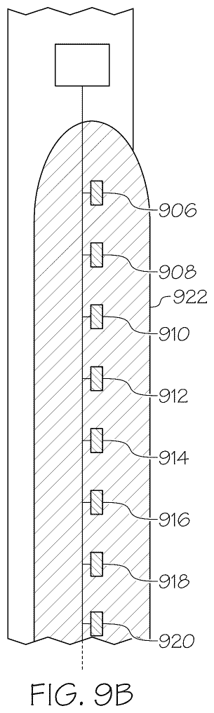

FIGS. 9A-9B are block diagrams of an eye-face tracking system in a near-eye device, in which the eye-face tracking sensors/projectors consist of an array of Self-Mixing Interferometric Vertical Cavity Side Emitting Lasers (SMINCSELs) which are capable of health monitoring, according to examples of the present disclosure. FIGS. 9A-9B show smaller partial cross-sections than in FIGS. 6-8B of the frame of a near-eye device in gray, within which there is a waveguide (in light blue). Unlike FIGS. 6-8B, the eye tracking system in FIGS. 9A-9B includes an array of Self-Mixing Interferometric Vertical Cavity Side Emitting Lasers (SMINCSELs), in canary, which are acting as both the eye/face tracking projectors and the eye/face tracking sensors.

In FIG. 9A, an array of eye/face tracking SMINCSELs projectors 902 (e.g., sensors) are are disposed adjacent to or as part of an example waveguide 904, which ultimately projects the light into an eye box (not shown) and receive the reflections of the speckle contrast patterns from an eye (through the example waveguide 904). As such, the array of eye/face tracking SMINCSELs projectors 902 in FIG. 9A may operate similar to the eye/face tracking cameras in FIGS. 8A-8B, except that, in addition to receiving the sparkle contrast reflections from the eye (as the eye/face tracking cameras in FIGS. 8A-8B do), the array of eye/face tracking SMINCSELs projectors 902 in FIG. 9A also project the sparkle contrast patterns into the eye box (indirectly by internal reflection through the waveguide)-a function which is performed by the eye/face tracking projectors in FIG. 8A-8B.

In FIG. 9B, the array of example eye/face tracking SMINCSELs projectors 906, 908, 910, 912, 914, 916 918, and 920 are represented as a series of individual SMINCSELs embedded directly in an example waveguide 922 and facing directly into an eye box (not shown). Unlike any of FIGS. 6-9A, the array of example eye/face tracking SMINCSELs projectors 906, 908, 910, 912, 914, 916 918, and 920 in FIG. 9B are pointed directly relative to an eye box from the eye lens, which may or may not also be a waveguide.

All of the descriptions above concerning FIGS. 6-8B apply equally to FIGS. 9A-9B, except that the aspects of the internal reflection (IR) in the waveguide in FIGS. 8A-8B apply only to FIG. 9A, and the aspect of eye/face tracking SMINCSELs projectors embedded directly in the eye lens apply only to FIG. 9B. Thus, for example, like the eye/face tracking cameras in FIGS. 8A-8B, the array of eye/face tracking SMINCSELs projectors in FIGS. 9A-9B may operate like a single unit or multiple separate units for the functions of projecting and/or sensing. For instance, the array of eye/face tracking SMINCSELs projectors in FIGS. 9A-9B may operate as either a global shutter eye/face tracking camera like in FIG. 6, or a rolling shutter eye/face tracking camera like in FIG. 7.

FIG. 10 is a block diagram of an example eye-face tracking system 1000 in a near-eye device, in which an example eye-face tracking camera 1002 is a high-resolution camera capable of health monitoring, according to examples of the present disclosure. In FIG. 10, the example eye-face tracking camera 1002 is pointed towards an example eye box such that it may take high resolution images of the user's eye. Unlike FIGS. 6-9B, no light source is shown in FIG. 10, because substantially any light source, including the ambient light of the environment, may serve as the light source of the user's eye when being imaged using the example eye-face tracking camera 1002 of FIG. 10 (e.g., the light source may be coherent or not coherent). In some examples, the example eye-face tracking camera 1002 of FIG. 10 is of a suitable resolution that it may register the microtremors caused by the user's heartbeat in the eye and/or surrounding facial tissues. In such examples, an example eye/face tracking controller 1004 may process the high-resolution images to determine, for example, the user's heart rate. Depending on context (as would be understood by one of ordinary skill in the art), all of the descriptions above concerning FIGS. 6-9B apply equally to FIG. 10, except that (1) the example eye-face tracking camera in FIG. 10 is a high-resolution camera; and (2) any available/suitable light source may be employed in FIG. 10 (including the light from the external environment) for the purposes of detecting microtremors caused by the user's heartbeat.

As mentioned above, in each of FIGS. 6-10, the eye/face tracking controller may constitute multiple components involved with performing eye/face tracking for the near-eye device. In some examples, the eye/face tracking controller may be the eye/face tracking unit 130 and/or the eye/face tracking module 118 of FIG. 1, or any other such eye/face tracking processing system. In some examples, the eye/face tracking controller may include a processor (not shown) and/or a memory (not shown), which may be a non-transitory computer-readable storage medium (and may store instructions executable by the processor and/or the eye/face tracking controller). In some examples, the eye/face tracking controller may perform any of the methods, functions, and/or processes described in any portion of the present disclosure by executing instructions contained on any suitable non-transitory computer-readable storage medium. In other examples, one or more other processors besides, or in addition to, the eye/face tracking controller, may perform any of the methods, functions, and/or processes described in any portion of the present disclosure by executing instructions contained on any suitable non-transitory computer-readable storage medium.

Below, non-limiting examples of methods for health monitoring using speckle contrast imaging which may be performed by the eye/face tracking controller (in purple) in FIGS. 6-10 and/or another suitable processor/controller are described.

FIG. 11 is a flowchart illustrating a method for an eye/face tracking system in a near-eye device to perform health monitoring using speckle contrast imaging, according to an example of the present disclosure. The method 1100 shown in FIG. 11 is provided by way of example and may only be one part of an entire process, procedure, ongoing operation, method, etc., as would be understood by one of ordinary skill in the art. The method 1100 may further omit parts of any process, procedure, ongoing operation, method, etc., involved in the method for an eye/face tracking system in a near-eye device to perform health monitoring using speckle contrast imaging not germane to examples of the present disclosure, as would be understood by one of ordinary skill in the art. Each block shown in FIG. 11 may further represent one or more steps, processes, methods, or subroutines, as would be understood by one of ordinary skill in the art. For the sake of convenience and ease of explanation, the blocks in FIG. 11 may refer to the components shown in the FIGS. described herein; however, the method 1100 is not limited in any way to the components, apparatuses, and/or constructions described and/or shown in any of the FIGS. herein. Some of the processes indicated by the blocks in FIG. 11 may overlap, occur substantially simultaneously, and/or be continually repeated.

At block 1110, one or more eye/face tracking projectors of an eye/face tracking system in a near-eye device illuminate the user's eye with a speckle pattern. In some examples, the eye/face tracking controller may control the one or more eye/face tracking projectors and may be any of the eye/face tracking controllers in any of FIGS. 6-9B. In some examples, the one or more eye/face tracking projectors may directly face the user's eye (e.g., FIGS. 6, 7, 8A, and/or 9B); in some examples, the one or more eye/face tracking projectors may project into, and reflect from, a hot mirror embedded in a display waveguide (e.g., FIG. 8B); in some examples, the one or more eye/face tracking projectors may project into a display waveguide and project therefrom into the user's eye, (e.g., FIG. 9A). In some examples, the one or more eye/face tracking projectors may include a multitude of eye/face tracking projectors in any configuration, disposition, and/or orientation (e.g., FIGS. 9A and 9B).

At block 1120, one or more eye/face tracking sensors of the eye/face tracking system in the near-eye device receive and sense the reflections of the projected speckle pattern from the user's eye. In some examples, the one or more eye/face tracking sensors may be any of the eye/face tracking cameras in FIGS. 6, 7, 8A, 8B, and/or 10; in other examples, the one or more eye/face tracking sensors may be the array of eye/face tracking SMINCSELs projectors/sensors in FIGS. 9A-9B. In some examples, the one or more eye/face tracking sensors may be either a global shutter eye/face tracking camera like in FIG. 6, or a rolling shutter eye/face tracking camera like in FIG. 7.

At block 1130, a time series of images/frames of the user's eye and/or surrounding facial tissue are created using data collected in block 1120. In some examples, the eye/face tracking controller (in purple) in any one or more of FIGS. 6-9B may generate the time series of images based on the reflections of the speckle pattern sensed/received by the one or more eye/face tracking sensors in block 1120. In other examples, a controller separate from the eye/face tracking system may generate the time series of images based on the reflections of the speckle pattern sensed/received by the one or more eye/face tracking sensors in block 1120 in accordance with examples of the present disclosure.

At block 1140, the eye/face tracking controller (in purple) in any one or more of FIGS. 6-9B may use the series of images obtained in block 1130 to perform speckle contrast imaging of the user's eye and/or surrounding tissue. In some examples, laser speckle contrast imaging (LSCI) or laser speckle imaging (LSI) may be employed to detect the motion of the blood flowing within the capillaries of the eye and/or surrounding tissue. In such examples, speckle statistics may be employed to determine, for example, where the surface capillaries are (by detecting the motion of the blood flowing within), thereby creating a map of the surface capillaries, and/or blood flow dynamics (hemodynamics) of the blood following in the capillaries. In some examples, such blood flow dynamics (hemodynamics) may include, for example, changes in blood flow within the capillaries; the viscosity of the blood plasma; the shapes and dynamics of the red blood cells; the osmotic pressure within the capillaries; hemodilution; the turbulence and velocity of blood flow; vascular resistance, stress, capacitance, and/or wall tension; and/or any other measurement/calculation which may be employed for health monitoring.

At block 1150, one or more processors operatively connected to (and/or part of) the eye/face tracking system in a near-eye device, such as the eye/face tracking controller (in purple) in any one or more of FIGS. 6-9B, may perform health monitoring using the results in block 1140 of the speckle contrast imaging of the user's eye and/or surrounding tissue. In some examples, the user's heart rate (pulse) may be determined in block 1240. In some examples, other types of health monitoring may be performed in block 1240 based on, e.g., the calculated/determined blood flow dynamics (hemodynamics) of the user's eye and/or surrounding tissue.

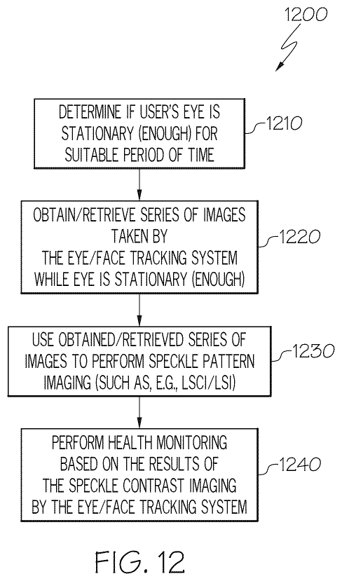

FIG. 12 illustrates a flow diagram for a method of health monitoring using speckle contrast imaging by an eye/face tracking system in a near-eye device, according to some examples. The method 1200 shown in FIG. 12 is provided by way of example and may only be one part of the entire process, procedure, technique, and/or method. The method 1200 may further omit parts of the process, procedure, technique, and/or method not germane to the present disclosure, as would be understood by one of ordinary skill in the art. Each block shown in FIG. 12 may further represent one or more steps, processes, methods, or subroutines, as would be understood by one of ordinary skill in the art. For the sake of convenience and ease of explanation, the description of the blocks in FIG. 12 may refer to the components of the near-eye device shown in one or more of the FIGS. above, although the method 1200 is not limited in any way to the components and/or construction of the near-eye devices in any of the FIGS. above. Some of the processes indicated by the blocks in FIG. 12 may overlap, occur substantially simultaneously, and/or be continually repeated.

At block 1210, one or more processors operatively connected to an eye/face tracking system in a near-eye device, such as the eye/face tracking controller in any one or more of FIGS. 6-10, may determine if the user's eye is and/or has been stationary (i.e., still, motionless, not currently moving) for a suitable period of time to perform speckle contrast imaging. In some examples, a controller/processor separate from the eye/face tracking system may determine whether the user's eye has been stationary for a suitable period of time. In some examples, the length of time the eye must be stationary may vary according to the specific components and parameters of the near-eye device being employed (e.g., the eye/face tracking sensor(s), the eye/face tracking projector(s), the eye/face tracking system architecture & configuration, etc.). In some examples, the length of time may depend on how many images the eye/face tracking camera(s) may take in a series in a certain amount of time. For instance, if the eye/face tracking camera(s) may take a few dozen images in less than a second while the eye is stationary, this may be adequate to perform the following steps in the method 1200. As mentioned herein, 10 to 100 milliseconds of stationary state of the eye (as indicated by, e.g., the location of the pupil) may be sufficient in some cases. In other cases, a small amount (e.g., a few degrees) of motion of the eyeball may be correctible by computer vision algorithms. Thus, such small movements may also be considered as stationary state.

In block 1220, one or more processors operatively connected to an eye/face tracking system in a near-eye device, such as the eye/face tracking controller in any one or more of FIGS. 6-10, may obtain and/or retrieve a series of images that were taken by the eye/face tracking system while the eye was stationary. In some examples, this may be done in real-time, i.e., as soon as the eye/face tracking controller determines the eye has been stationary for the appropriate period of time, the eye/face tracking controller may obtain the images which were already being taken by the eye tracking camera(s) to perform the following steps. In some examples, a controller separate from the eye/face tracking system may, after determining the user's eye has been stationary for the appropriate period of time, retrieve the series of images from the eye tracking camera(s) in accordance with examples of the present disclosure (or whatever storage unit is storing images from the eye tracking camera(s)).

At block 1230, one or more processors operatively connected to an eye/face tracking system in a near-eye device, such as the eye/face tracking controller in any one or more of FIGS. 6-10, may use the series of images obtained in block 1220 to perform speckle contrast imaging of the user's eye and/or surrounding tissue. In some examples, laser speckle contrast imaging (LSCI) or laser speckle imaging (LSI) may be employed to detect the motion of the blood flowing within the capillaries of the eye and/or surrounding tissue. In such examples, speckle statistics may be employed to determine, for example, where the surface capillaries are (by detecting the motion of the blood flowing within), thereby creating a map of the surface capillaries, and/or blood flow dynamics (hemodynamics) of the blood following in the capillaries. In some examples, such blood flow dynamics (hemodynamics) may include, for example, changes in blood flow within the capillaries; the viscosity of the blood plasma; the shapes and dynamics of the red blood cells; the osmotic pressure within the capillaries; hemodilution; the turbulence and velocity of blood flow; vascular resistance, stress, capacitance, and/or wall tension; and/or any other measurement/calculation which may be employed for health monitoring.

At block 1240, one or more processors operatively connected to an eye/face tracking system in a near-eye device, such as the eye/face tracking controller in any one or more of FIGS. 6-10, may perform health monitoring using the results in block 1230 of the speckle contrast imaging of the user's eye and/or surrounding tissue.

In some examples, the user's heart rate (pulse) may be determined in block 1240. In some examples, other types of health monitoring may be performed in block 1240 based on, e.g., the calculated/determined blood flow dynamics (hemodynamics) of the user's eye and/or surrounding tissue.

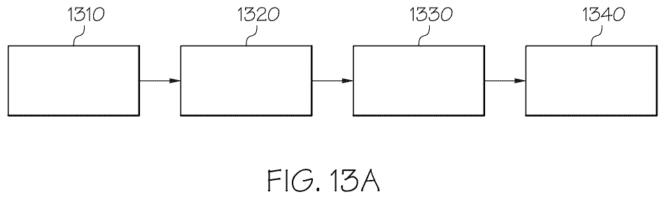

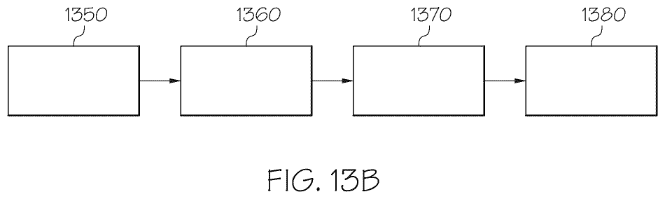

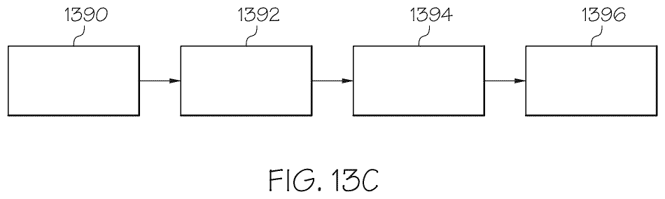

FIGS. 13A-13C are flowcharts illustrating methods for heart rate measurement using an eye/face tracking system in a near-eye device to perform speckle contrast imaging, according to examples of the present disclosure. The methods shown in FIGS. 13A-13C are provided by way of example and may only be one part of an entire process, procedure, ongoing operation, method, etc., as would be understood by one of ordinary skill in the art. The methods in FIGS. 13A-13C may further omit parts of any process, procedure, ongoing operation, method, etc., involved in the method for an eye/face tracking system in a near-eye device to perform heart rate measurement using speckle contrast imaging not germane to examples of the present disclosure, as would be understood by one of ordinary skill in the art. Each block shown in any of FIGS. 13A-13C may further represent one or more steps, processes, methods, or subroutines, as would be understood by one of ordinary skill in the art. For the sake of convenience and ease of explanation, the blocks in FIGS. 13A-13C may refer to the components shown in the FIGS. described herein; however, the methods in FIGS. 13A-13C are not limited in any way to the components, apparatuses, and/or constructions described and/or shown in any of the FIGS. herein. Some of the processes indicated by the blocks in methods in FIGS. 13A-13C may overlap, occur substantially simultaneously, and/or be continually repeated.

In FIGS. 13A-13B, a global shutter eye/face tracking camera like in FIG. 6 may be employed to create a time series of frames/images upon which the methods may be performed; whereas in FIG. 13C, a rolling shutter eye/face tracking camera like in FIG. 7 may be employed to generate visual data on a line-by-line basis. One or more processors operatively connected to (and/or part of) an eye/face tracking system in a near-eye device, such as the eye/face tracking controller (in purple) in any one or more of FIGS. 6-9B, may perform the methods shown in any of FIGS. 13A-13C.

In FIG. 13A, speckle contrast may be calculated globally within each frame at block 1310. At block 1320, the Fast Fourier Transform (FFT) of the average signal across each frame in the time series of frames/images may be calculated. At block 1330, the dominant frequency may be detected in the frequency domain, using the average signal FFT of block 1320. At block 1340, the heart rate may be measured, i.e., the pulse may be quantified, using the detected dominant frequency of block 1330.

In FIG. 13B, speckle contrast may be calculated globally within each frame at block 1350. At block 1360, the Fast Fourier Transform (FFT) of the average signal in the central portion of each frame (e.g., a box the size of 50×50 pixels) in the time series of frames/images may be calculated. At block 1370, the dominant frequency may be detected in the frequency domain, using the average signal FFT of block 1360. At block 1380, the heart rate may be measured, i.e., the pulse may be quantified, using the detected dominant frequency of block 1370.

In FIG. 13C, speckle contrast may be calculated line-by-line (i.e., a rolling shutter) of each frame at block 1390. At block 1392, the Fast Fourier Transform (FFT) of the temporally ordered values of calculated speckle contrast along each line may be calculated. At block 1394, the dominant frequency may be detected in the frequency domain, using the FFT values of block 1392. At block 1396, the heart rate may be measured, i.e., the pulse may be quantified, using the detected dominant frequency of block 1394.

As mentioned above, some of the processes indicated by the blocks in each of FIGS. 11 12, and 13A-13C may overlap, occur substantially simultaneously, and/or be continually repeated. Moreover, the methods described in FIGS. 11, 12, and 13A-13C are not mutually exclusive, but rather may be integrated into one another, overlap, occur substantially simultaneously (e.g., in parallel), and/or be continually repeated in roughly serial order.

As mentioned above, one or more processors may be employed in any near-eye display device to perform any of the methods, functions, and/or processes described herein by executing instructions contained on a non-transitory computer-readable storage medium. These one or more processors (such as, e.g., the one or more processors 121, the eye/face tracking unit 130, and/or the eye/face tracking module 118 in FIG. 1; the controller 317 (or one or more controller) of FIG. 3B, the eye/face tracking controller (in purple) in any one or more of FIGS. 6-10, and/or any other processing or controlling module which may be used in the near-eye display device in accordance with the present disclosure as would be understood by one of ordinary skill in the art), may be, or may include, one or more programmable general-purpose or special-purpose single- and/or multi-chip processors, a single- and/or multi-core processors, microprocessors, digital signal processors (DSPs), programmable controllers, application specific integrated circuits (ASICs), programmable logic device (PLDs), trust platform modules (TPMs), field-programmable gate arrays (FPGAs), other processing circuits, or a combination of these and other devices. Similarly, the non-transitory computer-readable storage medium which may contain those instructions for execution (such as, e.g., the application store 112 in optional console 110 of FIG. 1, any non-transitory computer-readable storage medium which may store instructions for the controller 317 (or one or more controllers) of FIG. 3B and/or the eye/face tracking controller (in purple) in any one or more of FIGS. 6-10, and/or any other storage module which may be used in the near-eye display device in accordance with the present disclosure as would be understood by one of ordinary skill in the art) may include read-only memory (ROM), flash memory, and/or random access memory (RAM)-any of which may be the main memory into which an operating system, various application programs, and/or a Basic Input-Output system (BIOS) which controls basic hardware operation such as the interaction with one or more peripheral components may be loaded/stored. Code or computer-readable instructions to implement the methods, functions, and/or operations discussed and/or described herein may be stored in any suitable computer-readable storage media and/or may be received via one or more communication/transmission interfaces, as would be understood by one of ordinary skill in the art.

According to examples, eye/face tracking systems, methods, and apparatuses in a near-eye device which may be employed for health monitoring are described herein. One or more methods for health monitoring utilizing the eye/face tracking system in a near-eye device are also described herein. A non-transitory computer-readable storage medium may have an executable stored thereon, which when executed instructs a processor to perform any of the methods described herein.

As discussed above, any of the controllers, processors, modules, components, integrated circuits, and/or processing units discussed and/or described herein may include one or more processors and one or more non-transitory computer-readable storage media storing instructions executable on such processors and/or any of the controllers, processors, modules, components, integrated circuits, and/or processing units discussed and/or described herein. In some examples, such processors and/or any of the controllers, processors, modules, components, integrated circuits, and/or processing units discussed and/or described herein may be implemented as hardware, software, and/or a combination of hardware and software in the near-eye display device. In some examples, such processors and/or the controllers, processors, modules, components, integrated circuits, and/or processing units discussed and/or described herein may be implemented, in whole or in part, by any type of application, program, library, script, task, service, process, or any type or form of executable instructions executed on hardware such as circuitry that may include digital and/or analog elements (e.g., one or more transistors, logic gates, registers, memory devices, resistive elements, conductive elements, capacitive elements, and/or the like, as would be understood by one of ordinary skill in the art).

In some examples, such processors and/or any of the controllers, processors, modules, components, integrated circuits, and/or processing units discussed and/or described herein may be implemented with a general purpose single- and/or multi-chip processor, a single- and/or multi-core processor, a digital signal processor (DSP), an application specific integrated circuit (ASIC), a field programmable gate array (FPGA), or other programmable logic device, discrete gate or transistor logic, discrete hardware components, and/or any combination thereof suitable to perform the functions described herein. A general purpose processor may be any conventional processor, microprocessor, controller, microcontroller, and/or state machine. In some examples, the one or more non-transitory computer-readable storage media may be implemented by one or more components (e.g., random access memory (RAM), read-only memory (ROM), flash or solid state memory, hard disk storage, etc.) for storing data and/or computer-executable instructions for completing and/or facilitating the processing and storage functions described herein. In such examples, the one or more non-transitory computer-readable storage media may be volatile and/or non-volatile memory, and may include database components, object code components, script components, or any other type of information structure suitable for implementing the various activities and storage functions described herein.

As would be understood by one of ordinary skill in the art, generally speaking, any one or more of the components and/or functionalities described in reference to any of the FIGS. herein may be implemented by hardware, software, and/or any combination thereof, according to examples of the present disclosure. In some examples, the components and/or functionalities may be implemented by any type of application, program, library, script, task, service, process, or any type or form of executable instructions stored in a non-transitory computer-readable storage medium executed on hardware such as circuitry that may include digital and/or analog elements (e.g., one or more transistors, logic gates, registers, memory devices, resistive elements, conductive elements, capacitive elements, and/or the like, as would be understood by one of ordinary skill in the art). In some examples, the hardware and data processing components used to implement the various processes, operations, logic, and circuitry described in connection with the examples described herein may be implemented with one or more of a general purpose single- and/or multi-chip processor, a single- and/or multi-core processor, a digital signal processor (DSP), an application specific integrated circuit (ASIC), a field programmable gate array (FPGA), or other programmable logic device, discrete gate or transistor logic, discrete hardware components, and/or any combination thereof suitable to perform the functions described herein.

A general purpose processor may be any conventional processor, microprocessor, controller, microcontroller, and/or state machine. In some examples, the memory/storage may include one or more components (e.g., random access memory (RAM), read-only memory (ROM), flash or solid state memory, hard disk storage, etc.) for storing data and/or computer-executable instructions for completing and/or facilitating the processing and storage functions described herein. In some examples, the memory/storage may be volatile and/or non-volatile memory, and may include database components, object code components, script components, or any other type of information structure suitable for implementing the various activities and storage functions described herein.

In the foregoing description, various examples are described, including devices, systems, methods, and the like. For the purposes of explanation, specific details are set forth in order to provide a thorough understanding of examples of the disclosure.

However, it will be apparent that various examples may be practiced without these specific details. For example, devices, systems, structures, assemblies, methods, and other components may be shown as components in block diagram form in order not to obscure the examples in unnecessary detail. In other instances, well-known devices, processes, systems, structures, and techniques may be shown without necessary detail in order to avoid obscuring the examples.

The figures/drawings and description are not intended to be restrictive. The terms and expressions that have been employed in this disclosure are used as terms of description and not of limitation, and there is no intention in the use of such terms and expressions of excluding any equivalents of the features shown and described or portions thereof. The word “example” is used herein to mean “serving as an example, instance, or illustration.” Any embodiment or design described herein as “example’ is not necessarily to be construed as preferred or advantageous over other embodiments or designs.

Clause 1. A device comprising: a frame including a projector and a sensor, wherein: the projector is configured to project a speckle pattern on a facial region of a user; and the sensor is configured to receive reflections of the speckled pattern; and a controller configured to: receive a time series of images of the facial region; and perform a speckle contrast imaging operation on the time series of images.

Clause 2. The device of clause 1, wherein the sensor includes a global shutter camera.

Clause 3. The device of clause 1, wherein the sensor includes a rolling shutter camera.

Clause 4. The device of clause 1, wherein the frame includes a waveguide.

Clause 5. The device of clause 1, wherein the projector disposed in a front portion of the frame.

Clause 6. The device of clause 4, wherein a hot mirror is disposed in the waveguide; and the projector disposed is in a temple of the frame.

Clause 7. The device of clause 4, wherein the projector includes an array of Self-Mixing Interferometric Vertical Cavity Side Emitting Lasers (SMINCSELs).

Clause 8. The device of clause 7, wherein the array is configured to illuminate a vasculature through the waveguide.

Clause 9. The device of clause 7, wherein the array of SMINCSELs is embedded in the waveguide.

Clause 10. The device of clause 1, wherein the controller is further configured to monitor, using results of the speckle contrast imaging operation, a cardiovascular parameter of the user.

Clause 11. A method comprising: projecting a speckle pattern on a facial region of a user; receiving, responsive to the projecting, reflections of the speckle pattern on the facial region; generating, using the reflections, a time series of images of the facial region of the user; and performing a speckle contrast imaging operation on the time series of images.

Clause 12. The method of clause 11, further comprising monitoring, using results of the speckle contrast imaging operation, a Cardiovascular parameter of the user.

Clause 13. The method of clause 12, wherein the cardiovascular parameter is a heart rate.

Clause 14. The method of clause 11, wherein the speckle contrast imaging operation is Laser Speckle Contrast Imaging (LSCI).

Clause 15. The method of clause 11, wherein the speckle contrast imaging operation is Laser Contrast Imaging (LCI).

Clause 16. A method comprising: projecting a speckle pattern on a facial region of a user; receiving, responsive to the projecting, reflections of the speckle pattern on the facial region; generating, using the reflections, a time series of images of the facial region of the user; determining whether the facial region is stationary for a time period; and performing, responsive to the determining, a speckle contrast imaging operation on the time series of images.

Clause 17. The method of clause 16, further comprising monitoring, using results of the speckle contrast imaging operation, a cardiovascular parameter of the user.

Clause 18. The method of clause 17, wherein the cardiovascular parameter is a heart rate.

Clause 19. The method of clause 16, wherein the speckle contrast imaging operation is Laser Speckle Contrast Imaging (LSCI).

Clause 20. The clause of claim 16, wherein the speckle contrast imaging operation is Laser Contrast Imaging (LCI).

Although the methods and systems as described herein may be directed mainly to digital content, such as videos or interactive media, it should be appreciated that the methods and systems as described herein may be used for other types of content or scenarios as well. Other applications or uses of the methods and systems as described herein may also include social networking, marketing, content-based recommendation engines, and/or other types of knowledge or data-driven systems.

What is claimed is:

1. A device comprising:
a frame including a projector and a sensor, wherein:
the projector is configured to project a speckle pattern on a facial region of a user; and
the sensor is configured to receive reflections of the speckled pattern; and
a controller configured to:
receive a time series of images of the facial region; and
perform a speckle contrast imaging operation on the time series of images.

2. The device of claim 1, wherein the sensor includes a global shutter camera.

3. The device of claim 1, wherein the sensor includes a rolling shutter camera.

4. The device of claim 1, wherein the frame includes a waveguide.

5. The device of claim 1, wherein the projector disposed in a front portion of the frame.

6. The device of claim 4, wherein:
a hot mirror is disposed in the waveguide; and
the projector is disposed in a temple of the frame.

7. The device of claim 4, wherein the projector includes an array of Self-Mixing Interferometric Vertical Cavity Side Emitting Lasers (SMINCSELs).

8. The device of claim 7, wherein the array is configured to illuminate a vasculature through the waveguide.

9. The device of claim 7, wherein the array of SMINCSELs is embedded in the waveguide.

10. The device of claim 1, wherein the controller is further configured to monitor, using results of the speckle contrast imaging operation, a cardiovascular parameter of the user.

11. A method comprising:
projecting a speckle pattern on a facial region of a user;
receiving, responsive to the projecting, reflections of the speckle pattern on the facial region;
generating, using the reflections, a time series of images of the facial region of the user; and
performing a speckle contrast imaging operation on the time series of images.

12. The method of claim 11, further comprising monitoring, using results of the speckle contrast imaging operation, a cardiovascular parameter of the user.

13. The method of claim 12, wherein the cardiovascular parameter is a heart rate.

14. The method of claim 11, wherein the speckle contrast imaging operation is Laser Speckle Contrast Imaging (LSCI).

15. The method of claim 11, wherein the speckle contrast imaging operation is Laser Contrast Imaging (LCI).

16. A method comprising:
projecting a speckle pattern on a facial region of a user;
receiving, responsive to the projecting, reflections of the speckle pattern on the facial region;
generating, using the reflections, a time series of images of the facial region of the user;
determining whether the facial region is stationary for a time period; and
performing, responsive to the determining, a speckle contrast imaging operation on the time series of images.

17. The method of claim 16, further comprising monitoring, using results of the speckle contrast imaging operation, a cardiovascular parameter of the user.

18. The method of claim 17, wherein the cardiovascular parameter is a heart rate.

19. The method of claim 16, wherein the speckle contrast imaging operation is Laser Speckle Contrast Imaging (LSCI).

20. The method of claim 16, wherein the speckle contrast imaging operation is Laser Contrast Imaging (LCI).
